(19) United States
(12) Patent Application Publication
(10) Pub. No.: US 2023/0159468 A1
(43) Pub. Date: May 25, 2023

(54) NOVEL FORMS OF PRACINOSTAT DIHYDROCHLORIDE

(71) Applicant: Macfarlan Smith Limited, Edinburgh (US)

(72) Inventor: Na YAO, Devens, MA (US)

(21) Appl. No.: 17/996,893

(22) PCT Filed: Apr. 21, 2021
(86) PCT No.: PCT/US2021/070438

§ 371 Date: Oct. 21, 2022

Related U.S. Application Data
(60) Provisional application No. 62/704,123, filed on Apr. 22, 2020.
(60) Provisional application No. 62/704,315, filed on May 4, 2020.

(51) Int. Cl. C07D 235/08

(52) U.S. Cl. CPC C07D 235/08; C07B 2200/13

(57) ABSTRACT

The present disclosure relates to various solid-state forms of pracinostat dihydrochloride, including a dimethyl sulfoxide solvate, an amorphous form, a hydrated form and another solid-state form, and several co-crystal forms, and to processes for their preparation. The present disclosure also relates to pharmaceutical compositions comprising any of these forms of pracinostat dihydrochloride and to their use in a method for treating a disease.

### FIELD OF THE DISCLOSURE

The present disclosure relates to various solid-state forms of pracinostat dihydrochloride, including a dimethyl sulfoxide solvate, an amorphous form, a hydrated form and another solid-state form, and several co-crystal forms, and to processes for their preparation. The present disclosure also relates to pharmaceutical compositions comprising any of these forms of pracinostat dihydrochloride and to their use in a method for treating a disease.

### BACKGROUND OF THE DISCLOSURE

Pracinostat, (E)-3-(2-Butyl-1-(2-(diethylamino)ethyl)-1H-benzo[d]imidazol-5-yl)-N-hydroxyacrylamide, is an oral histone deacetylase (HDAC) inhibitor that is being evaluated in a Phase 2 study in patients with high or very high-risk myelodysplastic syndromes (MDS). Both the U.S. Food and Drug Administration (FDA) and the European Medicine Agency (EMA) granted pracinostat in combination with azacytidine Orphan Drug Designation for newly diagnosed AML patients who are equal than or older than 75 years of age or not eligible for intensive chemotherapy. The FDA also granted the combination Breakthrough Therapy Designation. Pracinostat has the following structure:

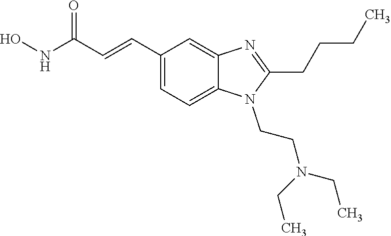 [SMILES: CCCCc1nc2cc(/C=C/C(=O)NO)ccc2n1CCN(CC)CC]

WO2019126282 discloses various forms of pracinostat free base (P1, P2, P3, P4, P6, P7, P8), a formic acid solvate, an acetic acid solvate, a butyric acid solvate, an iso-butyric acid solvate, Form II, Form 12, amorphous, and pracinostat sulfate salt Form S1. WO2019149262 discloses forms CS2 and CS5 of pracinostat free base. U.S. Patent Application Publication No. 2019/0152923 discloses the following forms of pracinostat dihydrochloride: Form 3 (2.5 hydrate); Form I (Monohydrate); Form 2 (Trihydrate); Form 4 (1.5 Hydrate); Form 5 (Anhydrous); Form 6 (limited stability); Form 7 (Hemi-Ethanol Solvate); Form 8 (wet solids); Form 9 (Anhydrous); and Form 10 (Hemihydrate). WO2018157741 discloses Forms CS7, CS9, CS1, and CS3 of pracinostat dihydrochloride. WO2018157742 discloses Form CS2 of pracinostat dihydrochloride. None of these references disclose a dimethyl sulfoxide solvate of pracinostat diHCl, an amorphous form of pracinostat diHCl, any pracinostat diHCl co-crystals, a hydrated form of pracinostat diHCl having peaks at 7.3, 9.2, 15.1, and 25.6° 2θ±0.2σ 2θ or a solid state form of pracinostat diHCl having peaks at 6.3, 7.7, 14.8, 19.5, 19.7 and 24.7σ 2θ±0.2σ 2θ.

### SUMMARY OF THE DISCLOSURE

The present invention is directed to various solid-state forms of pracinostat diHCl, including a dimethyl sulfoxide (DMSO) solvate, designated herein as Form A, an amorphous form, several co-crystal forms, namely, a pracinostat diHCl co-crystal with glycine, a pracinostat diHCl co-crystal with fumaric acid, and a pracinostat diHCl co-crystal with L-arginine, a hydrated form, designated as Form B, and another solid-state form, designated as Form C. The present invention is further directed to processes for the preparation of these forms. The present invention also is directed to pharmaceutical compositions comprising one or more of these forms and to their use in a method for treating disease.

### BRIEF DESCRIPTION OF THE DRAWINGS

FIG. 1 provides a representative XRPD pattern of Form A.

FIG. 2 provides a representative DSC plot of Form A.

FIG. 3 provides a representative TGA plot of Form A.

FIG. 4 provides a representative ¹H NMR of Form A in MeOH-d₄.

FIG. 5 provides a representative XRPD pattern of amorphous pracinostat diHCl.

FIG. 6 provides a representative mDSC plot of amorphous pracinostat diHCl.

FIG. 7 provides a representative ¹H NMR of amorphous pracinostat diHCl in DMSO-d₆.

FIG. 8 provides a representative XRPD pattern for a representative sample of the pracinostat diHCl co-crystal with glycine.

FIG. 9 provides a representative DSC plot of the pracinostat diHCl co-crystal with glycine.

FIG. 10 provides a representative TGA plot of the pracinostat diHCl co-crystal with glycine.

FIG. 11 provides a representative ¹H NMR of the pracinostat diHCl co-crystal with glycine in DMSO-d₆.

FIG. 12 provides a representative FTIR spectra of the pracinostat diHCl co-crystal with glycine.

FIG. 13 provides a representative FTIR spectra of pracinostat diHCl.

FIG. 14 provides a representative DVS plot of the pracinostat diHCl co-crystal with glycine.

FIG. 15 provides a representative XRPD pattern for a representative sample of the pracinostat diHCl co-crystal with fumaric acid.

FIG. 16 provides a representative DSC plot of the pracinostat diHCl co-crystal with fumaric acid.

FIG. 17 provides a representative TGA plot of the pracinostat diHCl co-crystal with fumaric acid.

FIG. 18 provides a representative ¹H NMR of the pracinostat diHCl co-crystal with fumaric acid in DMSO-d₆.

FIG. 19 provides a representative FTIR spectra of the pracinostat diHCl co-crystal with fumaric acid.

FIG. 20 provides a representative DVS plot of the pracinostat diHCl co-crystal with fumaric acid.

FIG. 21 provides a representative XRPD pattern for a representative sample of the pracinostat diHCl co-crystal with L-arginine.

FIG. 22 provides a representative DSC plot of the pracinostat diHCl co-crystal with L-arginine.

FIG. 23 provides a representative TGA plot of the pracinostat diHCl co-crystal with L-arginine.

FIG. 24 provides a representative ¹H NMR of the pracinostat diHCl co-crystal with L-arginine in D₂O.

FIG. 25 provides a representative FTIR spectra of the pracinostat diHCl co-crystal with L-arginine.

FIG. 26 provides a representative DVS plot of the pracinostat diHCl co-crystal with L-arginine.

FIG. 27 provides a representative XRPD pattern of Form B.

FIG. 28 provides a representative DSC plot of Form B.

FIG. 29 provides a representative TGA plot of Form B.

FIG. 30 provides a representative ¹H NMR of Form B in DMSO-d₆.

FIG. 31 provides a representative XRPD pattern of Form C.

FIG. 32 provides a representative ¹H NMR of Form C in DMSO-d₆.

### DETAILED DESCRIPTION OF THE DISCLOSURE

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles described herein may be applied to other examples and applications without departing from the spirit and scope of the various embodiments. Therefore, the various embodiments are not intended to be limited to the examples described herein and shown but are to be accorded the scope consistent with the claims.

### Terms/Definitions

As used herein and unless otherwise specified, the terms “about” and “approximately,” when used in connection with a numeric value or a range of values which is provided to characterize a particular solid form, e.g., a specific temperature or temperature range, such as, e.g., that describing a DSC or TGA thermal event, including, e.g., melting, dehydration, desolvation or glass transition events; a mass change, such as, e.g., a mass change as a function of temperature or humidity; a solvent or water content, in terms of, e.g., mass or a percentage; or a peak position, such as, e.g., in analysis by IR or Raman spectroscopy or XRPD; indicate that the value or range of values may deviate to an extent deemed reasonable to one of ordinary skill in the art while still describing the particular solid form.

As used herein and unless otherwise specified, the term “pharmaceutical composition” is intended to encompass a pharmaceutically effective amount of one or more of a DMSO solvate of pracinostat diHCl, an amorphous form of pracinostat diHCl, a pracinostat diHCl co-crystal with glycine, a pracinostat diHCl co-crystal with fumaric acid, a pracinostat diHCl co-crystal with L-arginine, Form B of pracinostat diHCl, and Form C of pracinostat diHCl, and a pharmaceutically acceptable excipient. As used herein, the term “pharmaceutical compositions” includes pharmaceutical compositions such as tablets, pills, powders, liquids, suspensions, emulsions, granules, capsules, suppositories, or injection preparations.

As used herein and unless otherwise specified, the term “crystalline” and related terms used herein, when used to describe a compound, substance, modification, material, component or product, unless otherwise specified, mean that the compound, substance, modification, material, component or product is substantially crystalline as determined by X-ray diffraction. See, e.g., Remington: The Science and Practice of Pharmacy, 21st edition, Lippincott, Williams and Wilkins, Baltimore, Md. (2005); The United States Pharmacopeia, 23rd ed., 1843-1844 (1995).

As used herein and unless otherwise specified, “co-crystal” and “co-crystal systems” refer to solid materials composed of two or more different components that are solid at room temperature and in particular stoichiometric ratios which interact through non-covalent interactions which can be designed utilizing supramolecular synthon approach. The co-crystal, in which at least one of the components is pracinostat diHCl and the coformer is a second pharmaceutically acceptable compound, is called a pharmaceutical pracinostat diHCl co-crystal with the coformer.

As used herein and unless otherwise specified, the term “excipient” refers to a pharmaceutically acceptable organic or inorganic carrier substance. Excipients may be natural or synthetic substances formulated alongside the active ingredient of a medication, included for the purpose of bulking-up formulations that contain potent active ingredients (thus often referred to as “bulking agents,” “fillers,” or “diluents”), or to confer a therapeutic enhancement on the active ingredient in the final dosage form, such as facilitating drug absorption or solubility. Excipients can also be useful in the manufacturing process, to aid in the handling of the active substance, such as by facilitating powder flowability or non-stick properties, in addition to aiding in vitro stability such as prevention of denaturation over the expected shelf life.

As used herein and unless otherwise specified, the term “patient” refers to an animal, preferably a mammal, most preferably a human, who has been the object of treatment, observation or experiment. Preferably, the patient has experienced and/or exhibited at least one symptom of the disease or disorder to be treated and/or prevented. Further, a patient may not have exhibited any symptoms of the disorder, disease or condition to be treated and/or prevented, but has been deemed by a physician, clinician or other medical professional to be at risk for developing said disorder, disease or condition.

As used herein and unless otherwise specified, the terms “polymorph,” “polymorphic form” or related term herein, refer to a crystalline form of an API (active pharmaceutical ingredient) free base or salt thereof that can exist in two or more forms, as a result of different arrangements or conformations of the molecule, ions of the salt, or addition and arrangement of solvents or coformers within the crystalline lattice of the crystal form.

As used herein and unless otherwise specified, the terms “substantially” or “substantially free/pure” with respect to a crystalline form means that the form contains about less than 30 percent, about less than 20 percent, about less than 15 percent, about less than 10 percent, about less than 5 percent, or about less than 1 percent by weight of impurities. Impurities may, for example, include other polymorphic forms, water and solvents other than that in a designated polymorphic form.

As used herein and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more therapeutic agents to a patient with such a disease or disorder. In some embodiments, the terms refer to the administration of a compound provided herein, with or without other additional active agents, after the onset of symptoms of the particular disease. Pracinostat is being studied in a Phase II trial in patients with high or very high-risk myelodysplastic syndromes (MDS).

As used herein and unless otherwise specified, the term “ambient temperature” refers to the working laboratory temperature range, about 18° C. to about 25° C.

As used herein and unless otherwise specified, the term “atmospheric pressure” refers to about 760 mm Hg.

It is therefore an object of the present disclosure to provide a pracinostat diHCl DMSO solvate, designated herein as Form A, an amorphous form of pracinostat diHCl, a pracinostat diHCl co-crystal with glycine, a pracinostat diHCl co-crystal with fumaric acid, a pracinostat diHCl co-crystal with L-arginine, a hydrated form of pracinostat diHCl, designated herein as Form B, and another solid-state form of pracinostat diHCl, designated herein as Form C, that are substantially pure, stable and scalable. It is also an object of the present disclosure to provide a pracinostat diHCl DMSO solvate, designated herein as Form A, an amorphous form of pracinostat diHCl, a pracinostat diHCl co-crystal with glycine, a pracinostat diHCl co-crystal with fumaric acid, a pracinostat diHCl co-crystal with L-arginine, a hydrated form of pracinostat diHCl, designated herein as Form B, another solid-state form of pracinostat diHCl, designated herein as Form C, that are capable of being isolated and handled.

Techniques for characterizing crystal and amorphous forms include but are not limited to differential scanning calorimetry (DSC), thermal gravimetric analysis (TGA), dynamic vapor sorption (DVS), X-ray powder diffractometry (XRPD), single crystal X-ray diffraction (SCXRD), proton nuclear magnetic resonance (¹H-NMR), Fourier transform infrared spectroscopy (FTIR Spectroscopy), and Optical Microscopy.

DSC data are collected using a TA Instruments Q2000 DSC. Approximately, samples (2-5 mg) are placed in sealed hermetic aluminum sample pans and scanned from about 25 to 350° C. at a rate of about 10° C./min under a nitrogen purge of 50 mL/min. The modulated DSC (mDSC) is carried out with modulation ±0.5° C. every 60 s and measured from about 5° C. to 120° C. at a heating rate of about 0.5° C./min under a nitrogen purge of about 50 mL/min.

TGA data are collected using a TA Instruments TGA Q500. Approximately, samples (2-5 mg) are placed in an open, pre-tared aluminum sample pan and scanned from about 25 to 350° C. at a rate of about 10° C./min using a nitrogen purge at about 60 mL/min.

XRPD patterns are obtained using a Bruker D8 Advance equipped with a Cu Kα radiation source (λ=1.54 Å), a 9-position sample holder and a LYNXEYE super speed detector. Samples are placed on zero-background, silicon plate holders for analysis. One skilled in the art would recognize that the °2θ values and the relative intensity values are generated by performing a peak search on the measured data and the d-spacing values are calculated by the instrument from the σ2θ values using Bragg's equation. One skilled in the art would further recognize that the relative intensity for the measured peaks may vary as a result of sample preparation, orientation and instrument used, for example.

¹H-NMR data are collected using a Bruker Ascend 600 MHz NMR equipped with TopSpin software. Samples are prepared by dissolving the compound in a deuterated solvent (DMSO-d₆, MeOH-d₄ or D₂O) with or without 0.05% (v/v) tetramethylsilane (TMS). Spectra are collected at 298 K.

The water content in a sample is measured by Mettler Toledo DL32 Karl Fisher (KF) coulometer. Solid samples (5-10 mg) are weighted into a weighing funnel that is used to transfer the material into the titration vessel. To limit the exposure of the solution to the air, the titrator opening is closed by a stopper immediately after adding samples. The samples are mixed for 30 seconds prior to analysis.

FTIR is analyzed using a Thermo Scientific Nicolet iS10. Approximately, samples (2-5 mg) with about 350 mg KBr are ground with a mortar and pestle, then pressed into pellets using a Little-Press KBr Pellet Die Kit. Each sample is measured with 16 scans in total within the range from about 4000 to 400 cm⁻¹.

In one embodiment, a DMSO solvate of pracinostat diHCl is prepared comprising:
a) combining pracinostat diHCl with a solvent selected from DMSO and a solvent mixture of DMSO and cyclohexane to obtain a solution of pracinostat diHCl; and
b) evaporating the solution of pracinostat diHCl to yield the DMSO solvate of pracinostat diHCl as a solid.

In one embodiment, the DMSO solvate of pracinostat diHCl is Form A. In one embodiment, the volume ratio of DMSO to cyclohexane in the solvent mixture is about 1:7-11; particularly about 1:9. Another embodiment optionally further comprises filtering the combination of pracinostat diHCl and solvent to ensure no particles remain, i.e., to obtain a clear solution of pracinostat diHCl. In one embodiment, the ratio of pracinostat diHCl to the solvent is not critical as long as a solution of pracinostat diHCl is formed; however, it is disadvantageous to use too dilute a solution as it will increase costs and the amount of time required for evaporation. In a particular embodiment, the weight to volume ratio of pracinostat diHCl to DMSO is about 150-175 mg 1 mL; particularly about 161 mg 1 mL, wherein if the pracinostat diHCl is not fully dissolved in the DMSO, the combination of pracinostat diHCl and DMSO is filtered to obtain a solution of pracinostat diHCl. In a particular embodiment, the weight to volume ratio of pracinostat diHCl to the solvent mixture of DMSO and cyclohexane is about 40-50 mg 1 mL; particularly about 46 mg 1 mL, wherein if the pracinostat diHCl is not fully dissolved in the solvent mixture, the combination of pracinostat diHCl and solvent mixture is filtered to obtain a solution of pracinostat diHCl. Another embodiment optionally further comprises applying heating during the combining or utilizing a solvent or solvent mixture that is heated. In another embodiment the heating occurs at about 50-70° C. (particularly about 60° C.), or the solvent or solvent mixture is heated to about 50-70° C. (particularly about 60° C.). In one embodiment, the solution of pracinostat diHCl is subjected to evaporation at room temperature. In yet another embodiment, the solution of pracinostat diHCl is subjected to evaporation at reduced pressure. The amount of time that the solution of pracinostat diHCl needs to be evaporated varies depending upon the volume of the solution of pracinostat diHCl and can take several days. The DMSO solvate of pracinostat diHCl; more particularly, Form A, can be dried by any conventional methods known to one of ordinary skill in the art.

In another embodiment, a DMSO solvate of pracinostat diHCl is prepared comprising:
a) combining pracinostat diHCl with a solvent mixture of DMSO and toluene to obtain a solution of pracinostat diHCl; and
b) adding additional toluene dropwise to the solution of pracinostat diHCl to yield the DMSO solvate of pracinostat diHCl as a solid.

In one embodiment, the DMSO solvate of pracinostat diHCl is Form A. In one embodiment, the volume ratio of DMSO to toluene in the solvent mixture is about 1:2-4; particularly about 1:3. Another embodiment optionally further comprises filtering the combination of pracinostat diHCl and solvent mixture of DMSO and toluene to ensure no particles remain, i.e., to obtain a clear solution of pracinostat diHCl. In one embodiment, the filtering occurs at room temperature. In one embodiment, the ratio of pracinostat diHCl to the solvent mixture is not critical as long as a solution of pracinostat diHCl is formed; however, it is disadvantageous to use too dilute a solution because the amount of additional toluene would also need to be increased. In a particular embodiment, the weight to volume ratio of pracinostat diHCl to the solvent mixture of DMSO and toluene is about 68-84 mg 1 mL; particularly about 76 mg 1 mL, wherein if the pracinostat diHCl is not fully dissolved in the solvent mixture of DMSO and toluene, the combination of pracinostat diHCl and solvent mixture is filtered to obtain a clear solution of pracinostat diHCl. In one embodiment, the combining occurs at room temperature. In one embodiment, the volume of additional toluene added to the solution is a volume sufficient to induce precipitation. In a particular embodiment, the volume ratio of the additional toluene to the solvent mixture of DMSO and toluene is about 3-5:1. In one embodiment, the method further comprises stirring the solvent mixture of DMSO and toluene after the adding of the additional toluene. In one embodiment, the stirring is at about 0-10° C.; particularly about 4° C. In one embodiment, the stirring is overnight (about 8-14 hr; particularly about 12 hr). The solid DMSO solvate of pracinostat diHCl can be isolated by any means known to one of skill in the art, e.g., decanting, vacuum filtration, and/or centrifugation. The DMSO solvate of pracinostat diHCl; more particularly, Form A, can be dried by any conventional methods known to one of ordinary skill in the art.

In another embodiment, a DMSO solvate of pracinostat diHCl is prepared comprising:
a) heating a mixture of pracinostat diHCl and a solvent mixture selected from DMSO/1,4-dioxane and DMSO/ethyl acetate to obtain a solution of the pracinostat diHCl; and
b) cooling the solution of the pracinostat diHCl to about 5° C. to yield the DMSO solvate of pracinostat diHCl as a solid.

In one embodiment, the DMSO solvate of pracinostat diHCl is Form A. In one embodiment, the volume ratio of DMSO to 1,4-dioxane in the solvent mixture is about 1:2-4; particularly about 1:3. In another embodiment, the volume ratio of DMSO to ethyl acetate in the solvent mixture is about 1:2-4; particularly about 1:3. Another embodiment optionally further comprises filtering the combination of pracinostat diHCl and solvent mixture to ensure no particles remain, i.e., to obtain a clear solution of pracinostat diHCl. In one embodiment, the ratio of pracinostat diHCl to the solvent mixture is not critical as long as a solution of pracinostat diHCl is formed; however, if too dilute a solution is used, the ratio of DMSO to 1,4-dioxane or the ratio of DMSO to ethyl acetate may need to be adjusted. In a particular embodiment, the weight to volume ratio of pracinostat diHCl to the solvent mixture of DMSO/1,4-dioxane is about 67-81 mg:1 mL; particularly about 74 mg:1 mL, wherein if the pracinostat diHCl is not fully dissolved in the solvent mixture of DMSO/1,4-dioxane, the combination of pracinostat diHCl and solvent mixture is filtered to obtain a solution of pracinostat diHCl. In a particular embodiment, the weight to volume ratio of pracinostat diHCl to the solvent mixture of DMSO/ethyl acetate is about 73-89 mg:1 mL; particularly about 81.5 mg:1 mL, wherein if the pracinostat diHCl is not fully dissolved in the solvent mixture of DMSO/ethyl acetate, the combination of pracinostat diHCl and solvent mixture is filtered to obtain a solution of pracinostat diHCl. In one embodiment, the heating is conducted at about 50-70° C.; particularly at about 60° C. In one embodiment, the cooling is at a rate of about 0.1° C./min. The solid DMSO solvate of pracinostat diHCl can be isolated by any means known to one of skill in the art, e.g., decanting, vacuum filtration, and/or centrifugation.

The DMSO solvate of pracinostat diHCl; more particularly, Form A, can be dried by any conventional methods known to one of ordinary skill in the art.
In another embodiment, amorphous pracinostat diHCl is prepared comprising: a) combining pracinostat diHCl with a solvent mixture of water and tert-butanol to form a solution;
b) shell freezing the pracinostat diHCl solution at about −78° C.; and
c) freeze drying the shell frozen pracinostat diHCl to yield the amorphous pracinostat diHCl.

In one embodiment, the volume ratio of water to tert-butanol in the solvent mixture is about 2.5-1.5:1; particularly about 2:1. In one embodiment, the combining is at about room temperature. In one embodiment, the ratio of pracinostat diHCl to the solvent mixture is not critical as long as a solution of pracinostat diHCl is formed. Another embodiment optionally further comprises filtering the combination of pracinostat diHCl and solvent mixture of water and tert-butanol to ensure no particles remain, i.e., to obtain a clear solution. In a particular embodiment, the weight to volume ratio of the pracinostat diHCl to the solvent mixture is about 116-142 mg:1 mL, particularly about 129 mg:1 mL, wherein if the pracinostat diHCl is not fully dissolved in the solvent mixture of water and tert-butanol, the combination of pracinostat diHCl and solvent mixture is filtered to obtain a solution of pracinostat diHCl. In one embodiment, the freeze drying occurs overnight (about 8-16 hr; particularly about 12 hr).

In another embodiment, a pracinostat diHCl co-crystal with glycine is prepared comprising:
a) suspending pracinostat diHCl in a solvent mixture of isopropyl alcohol and water;
b) suspending glycine in a solvent mixture of isopropyl alcohol and water, wherein the molar ratio of pracinostat diHCl to glycine is about 1:1;
c) mixing the pracinostat diHCl and glycine suspensions to yield a clear solution of pracinostat diHCl and glycine;
d) cooling the solution of pracinostat diHCl and glycine to yield the pracinostat diHCl co-crystal with glycine.

In one embodiment, the volume ratio of isopropyl alcohol to water in the solvent mixture is about 7-9:1, particularly about 8:1. In one embodiment, the weight to volume ratio of pracinostat diHCl to the solvent mixture is not critical as long as a suspension is formed. In a particular embodiment, the weight to volume ratio of pracinostat diHCl to the solvent mixture is about 45-55 mg:1 mL, particularly about 50 mg:1 mL. In one embodiment, the weight to volume ratio of glycine to the solvent mixture is not critical as long as a suspension is formed. In a particular embodiment, the weight to volume ratio of glycine to the solvent mixture is about 63-77 mg:1 mL, particularly about 70 mg:1 mL. In a particular embodiment, the solubility of the pracinostat diHCl in the solvent mixture is comparable to the solubility of the glycine in the solvent mixture, i.e., the weight % of the pracinostat diHCl dissolved in the solvent mixture is comparable to the weight % of the glycine dissolved in the solvent mixture. In one embodiment, the suspending and/or mixing is at about 35-45° C., particularly at about 40° C. In one embodiment, the mixing is for about 8-16 hr; particularly about 12 hr. In one embodiment, the cooling is to about 0-8° C.; particularly about 4° C. In one embodiment, the cooling rate is about 0.4-0.6° C./min, particularly about 0.5° C./min. The pracinostat diHCl co-crystal with glycine can be isolated by any means known to one of skill in the art, e.g., decanting, vacuum filtration, and/or centrifugation. The pracinostat diHCl co-crystal with glycine can be dried by any conventional methods known to one of ordinary skill in the art.

In another embodiment, a pracinostat diHCl co-crystal with fumaric acid is prepared comprising:
a) suspending pracinostat diHCl in a solvent mixture of isopropyl alcohol and water;
b) suspending fumaric acid in a solvent mixture of isopropyl alcohol and water, wherein the molar ratio of pracinostat diHCl to fumaric acid is about 1:1;
c) mixing the pracinostat diHCl and fumaric acid suspensions to yield a clear solution of pracinostat diHCl and fumaric acid;
d) cooling the solution of pracinostat diHCl and fumaric acid to yield the pracinostat diHCl co-crystal with fumaric acid.

In one embodiment, the volume ratio of isopropyl alcohol to water in the solvent mixture is about 7-9:1, particularly about 8:1. In one embodiment, the weight to volume ratio of pracinostat diHCl to the solvent mixture is not critical as long as a suspension is formed. In a particular embodiment, the weight to volume ratio of pracinostat diHCl to the solvent mixture is about 45-55 mg:1 mL, particularly about 50 mg:1 mL. In one embodiment, the weight to volume ratio of fumaric acid to the solvent mixture is not critical as long as a suspension is formed. In a particular embodiment, the weight to volume ratio of fumaric acid to the solvent mixture is about 97-119 mg:1 mL, particularly about 107.6 mg to about 1 mL. In a particular embodiment, the solubility of the pracinostat diHCl in the solvent mixture is comparable to the solubility of the fumaric acid in the solvent mixture, i.e., the weight % of the pracinostat diHCl dissolved in the solvent mixture is comparable to the weight % of the fumaric acid dissolved in the solvent mixture. In one embodiment, the suspending and/or mixing is at about 35-45° C., particularly about 40° C. In one embodiment, the mixing is for about 8-16 hr; particularly about 12 hr. In one embodiment, the cooling is to about 0-8° C.; particularly about 4° C. In one embodiment, the cooling rate is about 0.4-0.6° C./min, particularly about 0.5° C./min. The pracinostat diHCl co-crystal with fumaric acid can be isolated by any means known to one of skill in the art, e.g., decanting, vacuum filtration, and/or centrifugation. The pracinostat diHCl co-crystal with fumaric acid can be dried by any conventional methods known to one of ordinary skill in the art.

In another embodiment, a pracinostat diHCl co-crystal with fumaric acid is prepared comprising:
a) combining pracinostat diHCl with a solvent mixture of isopropyl alcohol and water at an elevated temperature to form a pracinostat diHCl solution;
b) adding solid fumaric acid to the pracinostat diHCl solution;
c) stirring the mixture of fumaric acid and pracinostat diHCl solution of step b at the elevated temperature;
d) cooling the mixture of step c to yield the pracinostat diHCl co-crystal with fumaric acid.

In one embodiment, the volume ratio of isopropyl alcohol to water in the solvent mixture is about 7-9:1, particularly about 8:1. In one embodiment, the weight to volume ratio of pracinostat diHCl to the solvent mixture is not critical as long as a solution is formed. In a particular embodiment, the weight to volume ratio of pracinostat diHCl to the solvent mixture is about 45-55 mg:1 mL, particularly about 50 mg:1 mL. Another embodiment optionally further comprises filtering the combination of pracinostat diHCl and solvent mixture to ensure no particles remain, i.e., to obtain a clear solution of pracinostat diHCl. In one embodiment, the molar ratio of pracinostat diHCl to fumaric acid is about 2:1. In another embodiment, the molar ratio of pracinostat diHCl to fumaric acid is about 1:1. In one embodiment, the elevated temperature is about 55-65° C., particularly about 60° C. In one embodiment, the stirring is for about 2-5 hr; particularly for about 3 hr. In one embodiment, the cooling is to about 0-10° C.; particularly to about 5° C. In one embodiment, the cooling rate is about 0.1° C./min. The pracinostat diHCl co-crystal with fumaric acid can be isolated by any means known to one of skill in the art, e.g., decanting, vacuum filtration, and/or centrifugation. The pracinostat diHCl co-crystal with fumaric acid can be dried by any conventional methods known to one of ordinary skill in the art.

In another embodiment, a pracinostat diHCl co-crystal with fumaric acid is prepared comprising:
a) combining pracinostat diHCl with a solvent mixture of isopropyl alcohol and water at an elevated temperature to form a pracinostat diHCl solution;
b) combining fumaric acid with a solvent mixture of isopropyl alcohol and water at the elevated temperature to form a fumaric acid solution, wherein the molar ratio of pracinostat diHCl to fumaric acid is about 1:1;
c) combining the pracinostat diHCl solution and the fumaric acid solution;
d) stirring the combined solution of step c at the elevated temperature;
e) cooling the combined solution to yield the pracinostat diHCl co-crystal with fumaric acid.

In one embodiment, the volume ratio of isopropyl alcohol to water in the solvent mixture is about 7-9:1, particularly about 8:1. In one embodiment, the weight to volume ratio of pracinostat diHCl to the solvent mixture is not critical as long as a solution is formed. In a particular embodiment, the weight to volume ratio of pracinostat diHCl to the solvent mixture is about 45-55 mg:1 mL, particularly about 50 mg:1 mL. In one embodiment, the weight to volume ratio of fumaric acid to the solvent mixture is not critical as long as a solution is formed. In a particular embodiment, the weight to volume ratio of fumaric acid to the solvent mixture is about 121-147 mg:1 mL, particularly about 134.5 mg:1 mL. Another embodiment optionally further comprises filtering the combination of pracinostat diHCl and solvent mixture to ensure no particles remain, i.e., to obtain a clear solution of pracinostat diHCl. Another embodiment optionally further comprises filtering the combination of fumaric acid and solvent mixture to ensure no particles remain, i.e., to obtain a clear solution of fumaric acid. In one embodiment, the elevated temperature is about 55-65° C., particularly about 60° C. In one embodiment, the stirring is for about 2-5 hr; particularly for about 3 hr. In one embodiment, the cooling is to about 0-10° C.; particularly to about 5° C. In one embodiment, the cooling rate is about 0.1° C./min. The pracinostat diHCl co-crystal with fumaric acid can be isolated by any means known to one of skill in the art, e.g., decanting, vacuum filtration, and/or centrifugation. The pracinostat diHCl co-crystal with fumaric acid can be dried by any conventional methods known to one of ordinary skill in the art.

In another embodiment, a pracinostat diHCl co-crystal with L-arginine is prepared comprising:
a) suspending pracinostat diHCl in a solvent mixture of isopropyl alcohol and water;
b) suspending L-arginine in a solvent mixture of isopropyl alcohol and water, wherein the molar ratio of pracinostat diHCl to L-arginine is about 1:1;
c) mixing the pracinostat diHCl and L-arginine suspensions to yield a clear solution of pracinostat diHCl and L-arginine; and
d) cooling the solution of pracinostat diHCl and L-arginine to yield the pracinostat diHCl co-crystal with L-arginine.

In one embodiment, the volume ratio of isopropyl alcohol to water in the solvent mixture is about 7-9:1, particularly about 8:1. In one embodiment, the weight to volume ratio of pracinostat diHCl to the solvent mixture is not critical as long as a suspension is formed. In a particular embodiment, the weight to volume ratio of pracinostat diHCl to the solvent mixture is about 45-55 mg:1 mL, particularly about 50 mg:1 mL. In one embodiment, the weight to volume ratio of glycine to the solvent mixture is not critical as long as a suspension is formed. In a particular embodiment, the weight to volume ratio of L-arginine to the solvent mixture is about 36-44 mg:1 mL, particularly about 40.3 mg to about 1 mL. In a particular embodiment, the solubility of the pracinostat diHCl in the solvent mixture is comparable to the solubility of the L-arginine in the solvent mixture, i.e., the weight % of the pracinostat diHCl dissolved in the solvent mixture is comparable to the weight % of the L-arginine dissolved in the solvent mixture. In one embodiment, the suspending and/or mixing is at about 35-45° C., particularly about 40° C. In one embodiment, the clear solution is obtained after about 5 minutes. In one embodiment, the mixing is for about 8-16 hr; particularly about 12 hr. In one embodiment, the precipitate forms after about 8-16 hr; particularly about 12 hr. In one embodiment, the cooling is to about 0-8° C.; particularly about 4° C. In one embodiment, the cooling rate is about 0.4-0.6° C./min, particularly about 0.5° C./min. The pracinostat diHCl co-crystal with L-arginine can be isolated by any means known to one of skill in the art, e.g., decanting, vacuum filtration, and/or centrifugation. The pracinostat diHCl co-crystal with L-arginine can be dried by any conventional methods known to one of ordinary skill in the art.

In one embodiment, Form B of pracinostat diHCl is prepared comprising:
a) combining pracinostat diHCl with a solvent mixture of isopropyl alcohol and water to obtain a solution of pracinostat diHCl;
b) adding Eudragit RS PO to the solution of pracinostat diHCl; and
c) cooling the solution of pracinostat diHCl with Eudragit RS PO of step b to yield Form B of pracinostat diHCl as a solid.

In one embodiment, the volume ratio of isopropyl alcohol to water in the solvent mixture is about 7-9:1; particularly about 8:1. In one embodiment, the solution of pracinostat diHCl is a saturated solution. The solubility of pracinostat diHCl in the solvent mixture is about 82.6 mg/mL at about 58° C. Another embodiment optionally further comprises filtering the combination of pracinostat diHCl and solvent mixture to ensure no particles remain, i.e., to obtain the solution of pracinostat diHCl. In a particular embodiment, the weight to volume ratio of pracinostat diHCl to the solvent mixture is about 84-104 mg:1 mL; particularly about 94 mg:1 mL, wherein if the pracinostat diHCl is not fully dissolved in the solvent mixture, the combination of pracinostat diHCl and solvent mixture is filtered to obtain the solution of pracinostat diHCl. In one embodiment, the weight to volume ratio of Eudragit RS PO to the solution of pracinostat diHCl is about 3.75-6.25 mg:1 mL. In one embodiment, the weight ratio of Eudragit RS PO to pracinostat diHCl is about 1 mg: 14-16 mg. Another embodiment further comprises applying heating during the combining or using a solvent mixture that is heated. In another embodiment, the heating occurs at about 50-70° C. (particularly about 60° C.), or the solvent mixture is heated to about 50-70° C. (particularly about 60° C.). In one embodiment, the cooling is to about 0-10° C., particularly to about 5° C. In one embodiment, the cooling is at a rate of about 0.05-0.2° C./min, particularly about 0.1° C./min. Form B of pracinostat diHCl can be isolated by any means known to one of skill in the art, e.g., decanting, vacuum filtration, and/or centrifugation. Form B can be dried by any conventional methods known to one of ordinary skill in the art.

In another embodiment, Form B of pracinostat diHCl is prepared comprising:
a) combining pracinostat diHCl with a solvent mixture of isopropyl alcohol and water to obtain a solution of pracinostat diHCl; and
b) evaporating the solution of the pracinostat diHCl to yield Form B of pracinostat diHCl as a solid.

In one embodiment, the volume ratio of isopropyl alcohol to water in the solvent mixture is about 7-9:1; particularly about 8:1. In one embodiment, the ratio of pracinostat diHCl to the solvent mixture is not critical as long as the solution of pracinostat diHCl is formed; however, the more dilute the solution, the longer the evaporating will take. Another embodiment optionally further comprises filtering the combination of pracinostat diHCl and solvent mixture to ensure no particles remain, i.e., to obtain the solution of pracinostat diHCl. In a particular embodiment, the weight to volume ratio of pracinostat diHCl to the solvent mixture is about 84-104 mg:1 mL; particularly about 94 mg:1 mL, wherein if the pracinostat diHCl is not fully dissolved in the solvent mixture, the combination of pracinostat diHCl and solvent mixture is filtered to obtain the solution of pracinostat diHCl. Another embodiment further comprises applying heating during the combining or using a solvent mixture that is heated. In another embodiment, the heating occurs at about 50-70° C. (particularly about 60° C.), or the solvent mixture is heated to about 50-70° C. (particularly about 60° C.). In one embodiment, the solution of pracinostat diHCl is subjected to evaporation at room temperature. In yet another embodiment, the solution of pracinostat diHCl is subjected to evaporation at reduced pressure. In a further embodiment, the evaporating of the solution of the pracinostat diHCl is under a fume hood. The amount of time that the solution of pracinostat diHCl needs to be evaporated varies depending upon the volume of the solution of pracinostat diHCl; particularly, evaporation can take several days. Form B of pracinostat diHCl can be dried by any conventional methods known to one of ordinary skill in the art.

In another embodiment, Form B of pracinostat diHCl is prepared comprising:
a) combining pracinostat diHCl with water to obtain a solution of pracinostat diHCl; and
b) adding dioxane dropwise to the solution of pracinostat diHCl to yield Form B of pracinostat diHCl as a solid.

In one embodiment, the ratio of pracinostat diHCl to water is not critical as long as the solution of pracinostat diHCl is formed; however, it is disadvantageous to use too dilute a solution because the amount of dioxane would need to be increased. Another embodiment optionally further comprises filtering the combination of pracinostat diHCl and water to ensure no particles remain, i.e., to obtain the solution of pracinostat diHCl. In a particular embodiment, the weight to volume ratio of pracinostat diHCl to the water is about 130-158 mg:1 mL; particularly about 144 mg:1 mL, wherein if the pracinostat diHCl is not fully dissolved in the water, the combination of pracinostat diHCl and water is filtered to obtain the solution of pracinostat diHCl. In one embodiment, the volume of dioxane added to the solution of pracinostat diHCl is a volume sufficient to induce precipitation. In a particular embodiment, the volume ratio of the dioxane to the solution of pracinostat diHCl is about 9-11 mL:1 mL, particularly about 10 mL:1 mL. In one embodiment, the method further comprises stirring the solution of the pracinostat diHCl while adding the dioxane. In one embodiment, the stirring is at about 0-10° C.; particularly about 4° C. Form B can be isolated by any means known to one of skill in the art, e.g., decanting, vacuum filtration, and/or centrifugation. Form B can be dried by any conventional methods known to one of ordinary skill in the art.

In another embodiment, Form C of pracinostat diHCl is prepared comprising:
a) combining pracinostat diHCl with methanol to obtain a solution of pracinostat diHCl; and
b) allowing vapor diffusion to occur between dichloromethane and the solution of pracinostat diHCl to yield Form C of pracinostat diHCl as a solid.

In one embodiment, the combining occurs at about room temperature. In one embodiment, the pracinostat diHCl solution is saturated or nearly saturated. If the solution is dilute, the solution appears colorless. If the solution is at or near saturation, the solution appears brownish, but transparent, in color. Another embodiment optionally further comprises filtering the combination of pracinostat diHCl and methanol to ensure no particles remain, i.e., to obtain the solution of pracinostat diHCl. In a particular embodiment, the weight to volume ratio of the pracinostat diHCl to the methanol is about 135-165 mg:1 mL, particularly about 150 mg:1 mL, wherein if the pracinostat diHCl is not fully dissolved in the methanol, the combination of pracinostat diHCl and methanol is filtered to obtain the solution of pracinostat diHCl. In one embodiment, the volume ratio of the solution of pracinostat diHCl to dichloromethane is about 1 mL:10-15 mL, particularly about 1 mL:12.5 mL. In one embodiment, the allowing vapor diffusion to occur comprises placing a volume of the pracinostat diHCl solution in an open container; and placing the open container inside another container (which is closed) containing a volume of dichloromethane thereby allowing dichloromethane vapor to interact with the solution of pracinostat diHCl. In one embodiment, the solid is precipitated after several weeks, particularly about 3 weeks. Form C can be isolated by any means known to one of skill in the art, e.g., decanting, vacuum filtration, and/or centrifugation. Form C can be dried by any conventional methods known to one of ordinary skill in the art.

An embodiment of the invention is directed to Form A that is a DMSO solvate of pracinostat diHCl wherein the eq ratio of pracinostat diHCl to DMSO is about 1:2. Another embodiment of the invention is an amorphous form of pracinostat diHCl. A further embodiment of the invention is directed to a pracinostat diHCl co-crystal, particularly the pracinostat diHCl co-crystal with glycine existing in a 1:1 eq ratio, a pracinostat diHCl co-crystal with fumaric acid existing in a 2:1 eq ratio, and a pracinostat diHCl co-crystal with L-arginine existing in a 1:1 eq ratio. A further embodiment of the invention is directed to Form B which is a hydrated form of pracinostat diHCl. An additional embodiment of the invention is directed to Form C which is a solid-state form of pracinostat diHCl. A further embodiment of the invention is directed to any of these forms prepared by a process embodiment as described herein.

The present disclosure also encompasses a pharmaceutical composition comprising one or more of a DMSO solvate of pracinostat diHCl, an amorphous form of pracinostat diHCl, a pracinostat diHCl co-crystal with glycine, a pracinostat diHCl co-crystal with fumaric acid, a pracinostat diHCl co-crystal with L-arginine, Form B of pracinostat diHCl, and Form C of pracinostat diHCl; and a pharmaceutically acceptable excipient. The pharmaceutical composition may be prepared according to U.S. Pat. No. 9,717,713 or any other methods known in the art.

The present disclosure provides for a method of treating disease by administering to a patient in need thereof a pharmaceutical composition comprising one or more of a DMSO solvate of pracinostat diHCl, an amorphous form of pracinostat diHCl, a pracinostat diHCl co-crystal with glycine, a pracinostat diHCl co-crystal with fumaric acid, a pracinostat diHCl co-crystal with L-arginine, Form B of pracinostat diHCl, and Form C of pracinostat diHCl. Pracinostat is being studied in a Phase II trial in patients with high or very high-risk myelodysplastic syndromes (MDS). Pracinostat may also be useful in treating inflammatory disorders, immune system disorders, cardiovascular diseases, fibrotic diseases, vascular diseases, viral diseases, neurological diseases, parasitic diseases and proliferative disorders. Other uses may include colon cancer, ovarian cancer, prostate cancer, breast cancer, lung cancer, liver cancer, pancreatic cancer, renal cancer, sarcoma, neuroblastoma, gastric cancer, multiple myeloma, myeloproliferative neoplasms, and hematologic cancers/malignancies.

The dosage of the pharmaceutical compositions may be varied over a wide range. Optimal dosages and dosage regimens to be administered may be readily determined by those skilled in the art, and will vary with the mode of administration, the strength of the preparation and the advancement of the disease condition. In addition, factors associated with the particular patient being treated, including patient's sex, age, weight, diet, physical activity, time of administration and concomitant diseases, will result in the need to adjust dosages and/or regimens.

### Examples

Examples 1-16 which follow herein provide exemplary embodiments of the various solid-state forms of pracinostat diHCl.

The Examples are presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles described herein may be applied to other examples and applications without departing from the spirit and scope of the various embodiments. Therefore, the various embodiments are illustrative of the present disclosure and the disclosure is not intended to be limited to the examples described herein and shown.

### Example 1—Preparation of Pracinostat diHCl DMSO Solvate

Step A

56.2 mg (0.13 mmol) of a low crystallinity pracinostat diHCl (likely a mixture of amorphous and a crystalline form) is slurried in 1 mL pure cyclohexane at 60° C. for three days. A solid sample is isolated for XRPD analysis which indicates that the sample is amorphous.

Step B

The wet amorphous solid from Step A is subjected to ultrasonication (35 kHz) at 30° C. for 45 min. A solid is isolated for XRPD analysis which indicates that the sample is still amorphous.

Step C

0.1 mL DMSO is added to the wet solid from Step B (solid weight is ˜46.2 mg, 0.11 mmol) and the mixture is heated to 60° C. The final solvent system is 0.1 mL DMSO and ˜0.9 mL cyclohexane. Temperature cycling is applied to the sample. Specifically, the sample is cooled from 60° C. to 5° C. at the cooling rate of 0.1° C./min. The sample is then heated up to 60° C. at the heating rate of 5° C./min and cooled again from 60° C. to 5° C. at the cooling rate of 0.1° C./min resulting in a clear pink solution at 5° C.

Step D

The solution from Step C is evaporated at RT. The solid obtained from evaporation is analyzed by XRPD and determined to be a DMSO solvate of pracinostat diHCl, particularly, Form A. KF measurement of Form A indicates the presence of residual water at about 0.4%.

Form A is characterized by its XRPD pattern peaks. 20 and relative % intensity values for peaks are shown in Table 1.

TABLE 1
Average Peak List for Form A diffractogram

| Angle | |
|---|---|
| 2-Theta ° | Intensity % |
| 6.5 | 45.1 |
| 7.6 | 34.9 |
| 8.9 | 12.2 |
| 9.6 | 23.1 |
| 9.7 | 22.7 |
| 9.9 | 13.6 |
| 10.5 | 15.6 |
| 12.1 | 14.2 |
| 14.7 | 23.7 |
| 15.6 | 10.5 |
| 15.9 | 20.0 |
| 16.9 | 11.9 |
| 18.1 | 45.1 |
| 18.7 | 100.0 |
| 19.3 | 27.1 |
| 19.6 | 43.1 |
| 20.6 | 51.9 |
| 21.0 | 27.1 |
| 21.4 | 33.9 |
| 21.7 | 37.6 |
| 22.1 | 35.3 |
| 22.6 | 14.2 |
| 23.3 | 35.3 |
| 23.6 | 19 |
| 24.2 | 71.2 |
| 24.5 | 24.7 |
| 24.8 | 41.4 |
| 25.3 | 8.1 |
| 25.9 | 19.3 |
| 26.3 | 46.4 |
| 26.8 | 22.7 |
| 27.1 | 57.3 |
| 27.7 | 9.5 |
| 28.0 | 14.2 |
| 28.5 | 22.4 |
| 29.0 | 10.5 |
| 29.3 | 23.1 |
| 29.7 | 13.6 |
| 30.4 | 8.8 |
| 30.8 | 8.8 |
| 31.4 | 21.4 |
| 31.7 | 13.6 |
| 33.0 | 10.8 |
| 33.9 | 10.2 |
| 35.9 | 9.8 |
| 36.8 | 13.6 |
| 37.5 | 9.5 |
| 38.6 | 7.5 |

The angle measurements are ±0.2° 2θ. In one embodiment, key defining peaks for Form A include two or more of 18.7, 24.2, and 27.1° 2θ. In another embodiment, key defining peaks for Form A further comprise one or more peaks selected from 6.5, 18.1, 20.6, and 26.3° 2θ.

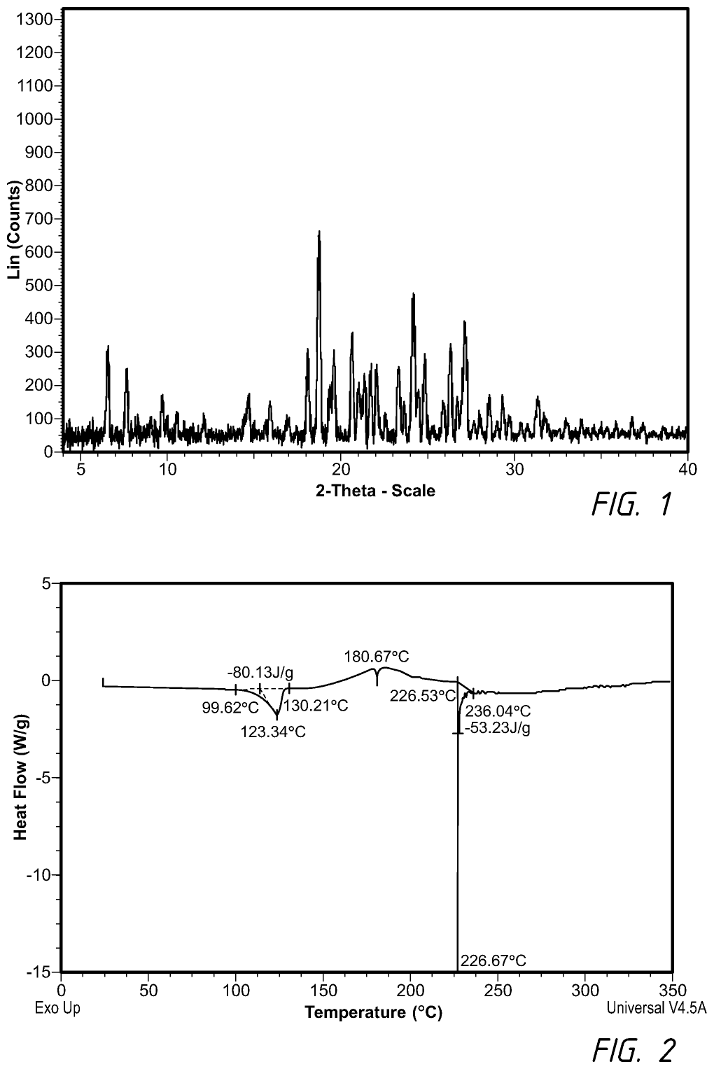

FIG. 1 provides a representative XRPD pattern for a representative sample of Form A.

FIG. 2 provides a representative DSC plot of Form A that shows the onset of two endothermic events, one at about 100° C. and the other at about 227° C., and a small endothermic event at about 181° C.

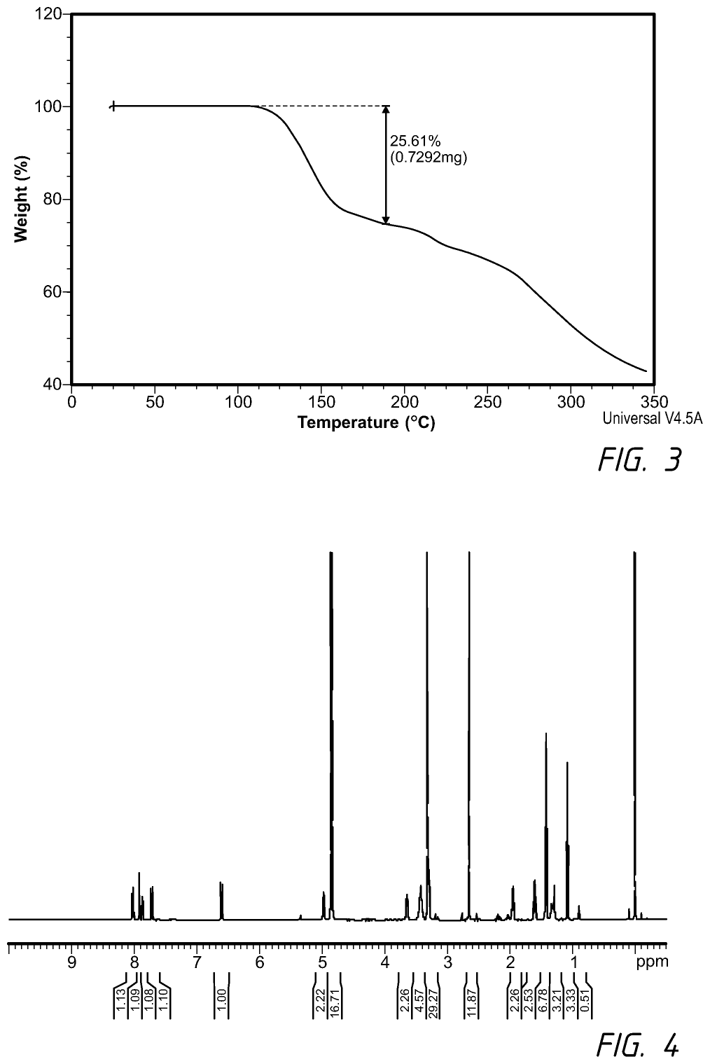

FIG. 3 provides a representative TGA plot of Form A that shows a weight loss of about 25.6% up to about 189° C. (the boiling point of DMSO).

FIG. 4 provides a representative ¹H NMR of Form A. Based on the integration of 1H of pracinostat diHCl at 6.6 ppm and 6H of DMSO at 2.7 ppm, the molar ratio of pracinostat diHCl to DMSO is determined to be 1:2. The weight percentage of DMSO is about 26.6%, which is close to the weight loss that occurred by TGA.

### Example 2—Preparation of Pracinostat diHCl DMSO Solvate

321.5 mg (0.74 mmol) of pracinostat diHCl is dissolved in 2 mL DMSO, resulting in a clear solution. The solution is filtered to ensure no particles remain. The solution is kept in an open vial and covered with parafilm with 6 pin holes for evaporation at room temperature. The solid is isolated by evaporation and is analyzed by XRPD and determined to be a DMSO solvate of pracinostat diHCl, particularly, Form A.

### Example 3—Preparation of Pracinostat diHCl DMSO Solvate

76.3 mg (0.18 mmol) pracinostat diHCl is combined with 1 mL of a solvent mixture of DMSO and toluene (1:3, v/v). The sample is filtered to obtain a clear solution at room temperature. Toluene is added to the clear solution dropwise. Precipitation is observed after 3 mL toluene is added. Another 2 mL toluene is added (a total of 5 mL toluene) and the sample is stirred overnight at 4° C. The solid is isolated by vacuum filtration. XRPD analysis indicates that the solid is a DMSO solvate of pracinostat diHCl, particularly, Form A.

### Example 4—Preparation of Pracinostat diHCl DMSO Solvate

74.0 mg (0.17 mmol) pracinostat diHCl is combined with 1 mL of a solvent mixture of DMSO and 1,4-dioxane (1:3, v/v) at 60° C. and filtered to obtain a clear solution. The clear solution is cooled from 60° C. to 5° C. at the cooling rate of 0.1° C./min, resulting in a solid. The solid is isolated and analyzed by XRPD which indicates that the solid is a DMSO solvate of pracinostat diHCl, particularly, Form A.

### Example 5—Preparation of Pracinostat diHCl DMSO Solvate

81.5 mg (0.19 mmol) pracinostat diHCl is combined with 1 mL of a solvent mixture of DMSO and ethyl acetate (1:3, v/v) at 60° C. and filtered to obtain a clear solution. The clear solution is cooled from 60° C. to 5° C. at the cooling rate of 0.1° C./min, resulting in a solid. The solid is isolated and analyzed by XRPD which indicates that the solid is a DMSO solvate of pracinostat diHCl, particularly, Form A.

### Example 6—Preparation of Amorphous Pracinostat diHCl

193.6 mg (0.45 mmol) pracinostat diHCl is dissolved in 1.5 mL of a solvent mixture of water and tert-butanol (2:1, v/v) at room temperature. The pracinostat diHCl solution is shell frozen in an acetone bath with dry ice. The frozen sample is connected to a freeze dryer and dried overnight, resulting in amorphous pracinostat diHCl.

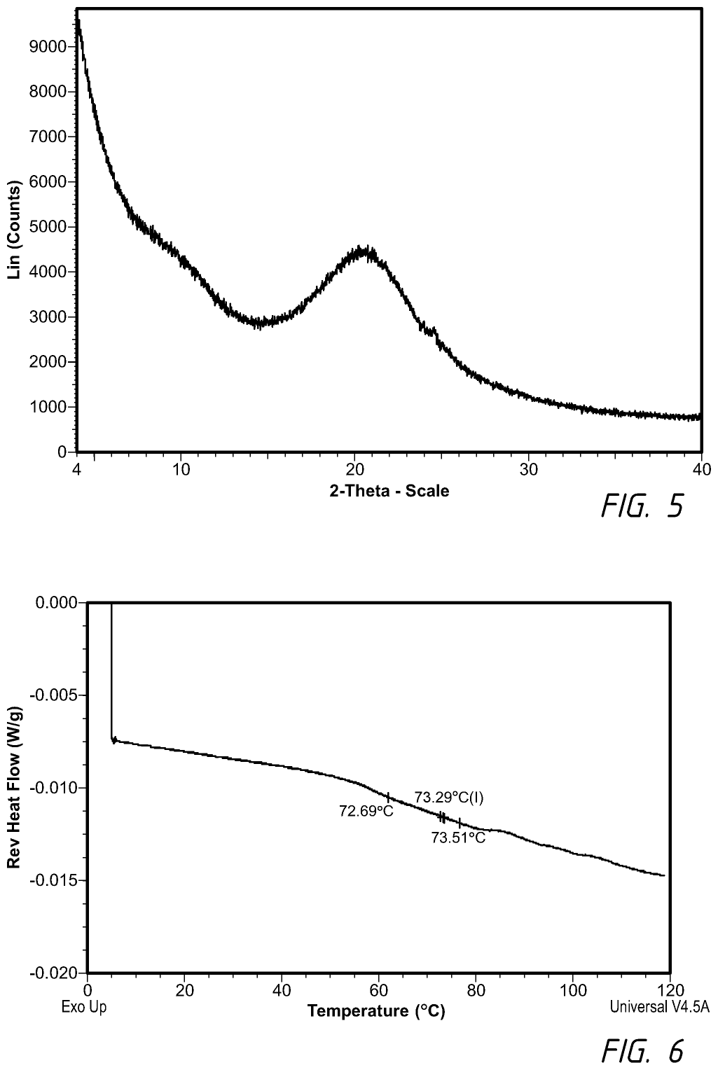

FIG. 5 provides a representative XRPD pattern for a representative sample of amorphous pracinostat diHCl.

FIG. 6 provides a representative modulated DSC plot of amorphous pracinostat diHCl which shows the glass transition temperature to be about 73° C.

FIG. 7 provides a representative ¹H NMR of amorphous pracinostat diHCl which shows that no residual solvent is present in the sample.

### Example 7—Preparation of Pracinostat diHCl Co-Crystal with Glycine

100 mg (0.23 mmol) of pracinostat diHCl is suspended in 2 mL of a solvent mixture of isopropyl alcohol (IPA) and water (8:1, v/v) at 40° C. 17.4 mg (0.23 mmol) glycine is suspended in 5 drops (˜0.25 mL) of a solvent mixture of IPA and water (8:1, v/v) at 40°. The pracinostat diHCl and glycine samples are combined and stirred at 40° C. overnight (˜12 hr), resulting in a clear solution. The clear solution is cooled from 40° C. to 4° C. at the cooling rate of 0.5° C./min, resulting in a frozen solid. The frozen solid is transferred to room temperature causing the solid to melt. The solid is isolated by vacuum filtration and analyzed by XRPD and determined to be a pracinostat diHCl co-crystal with glycine.

KF measurement indicates the presence of about 4.2% (weight) water in the pracinostat diHCl co-crystal with glycine, which suggests it is a hydrate, more likely, a monohydrate.

The pracinostat diHCl co-crystal with glycine is characterized by its XRPD pattern peaks. 2θ and relative % intensity values for peaks are shown in Table 2.

TABLE 2
Average Peak List for Pracinostat diHCl
Co-crystal with Glycine Diffractogram

| Angle | Intensity |
|---|---|
| 2-Theta ° | % |
| 7.5 | 36.1 |
| 8.6 | 10.4 |
| 9.2 | 10.0 |
| 10.4 | 10.7 |
| 11.3 | 14.3 |
| 12.4 | 9.3 |
| 14.3 | 12.5 |
| 15.1 | 28.2 |
| 16.5 | 10.7 |
| 17.4 | 17.5 |
| 21.6 | 13.9 |
| 23.1 | 23.6 |
| 25.0 | 100.0 |
| 25.7 | 8.9 |
| 29.0 | 30.7 |
| 29.6 | 13.6 |
| 30.6 | 8.6 |
| 38.9 | 31.1 |

The angle measurements are ±0.2° 2θ. In one embodiment, key defining peaks for the pracinostat diHCl co-crystal with glycine include two or more of 7.5, 25.0, 29.0, and 38.9° 2θ. In another embodiment, key defining peaks for the pracinostat diHCl co-crystal with glycine further comprise one or more peaks selected from 15.1 and 23.1° 2θ.

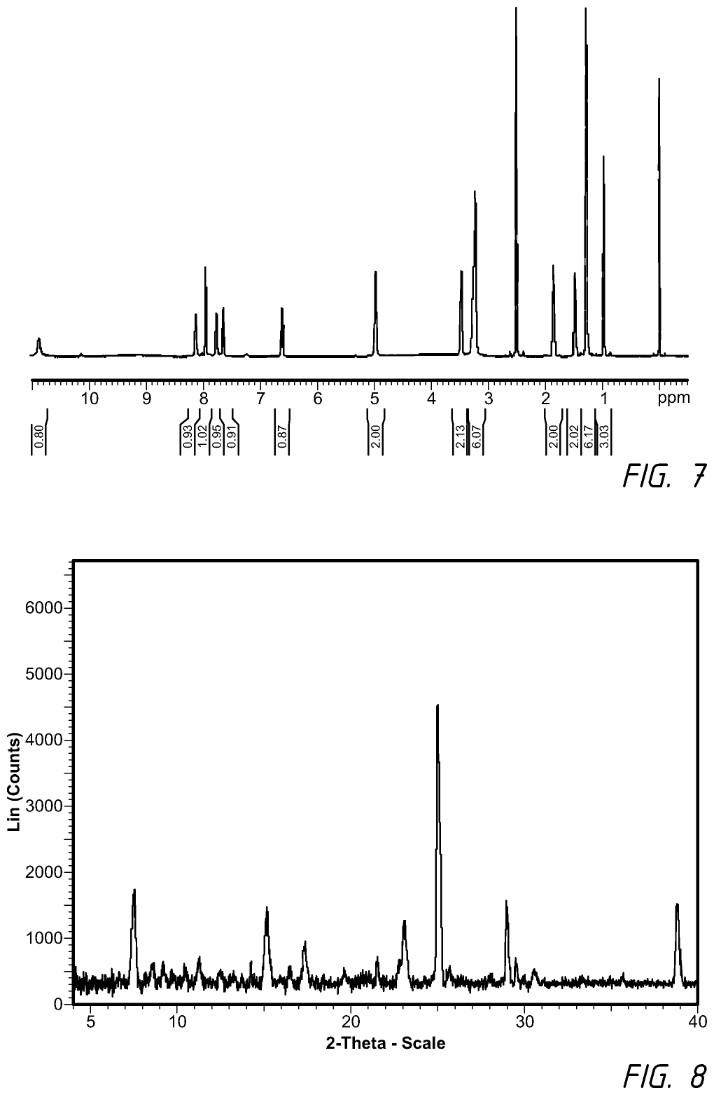

FIG. 8 provides a representative XRPD pattern for a representative sample of the pracinostat diHCl co-crystal with glycine.

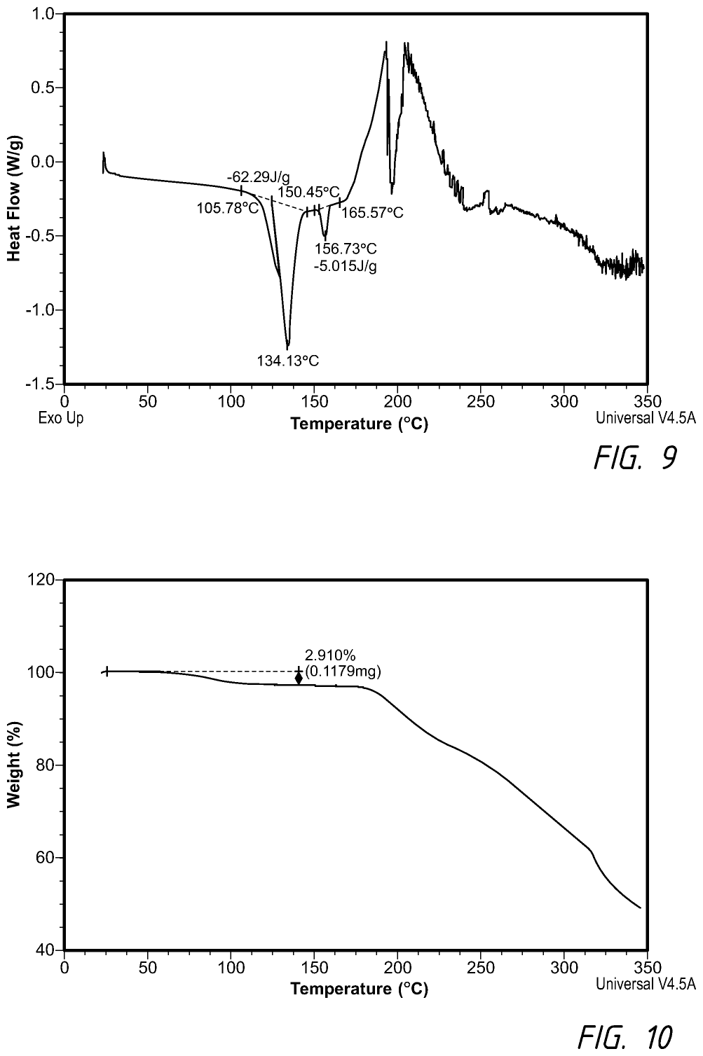

FIG. 9 provides a representative DSC plot of the pracinostat diHCl co-crystal with glycine that shows the onset of two endothermic events, one at about 106° C. and another at about 150° C.

FIG. 10 provides a representative TGA plot of the pracinostat diHCl co-crystal with glycine that shows a weight loss of about 3.0% up to about 140° C.

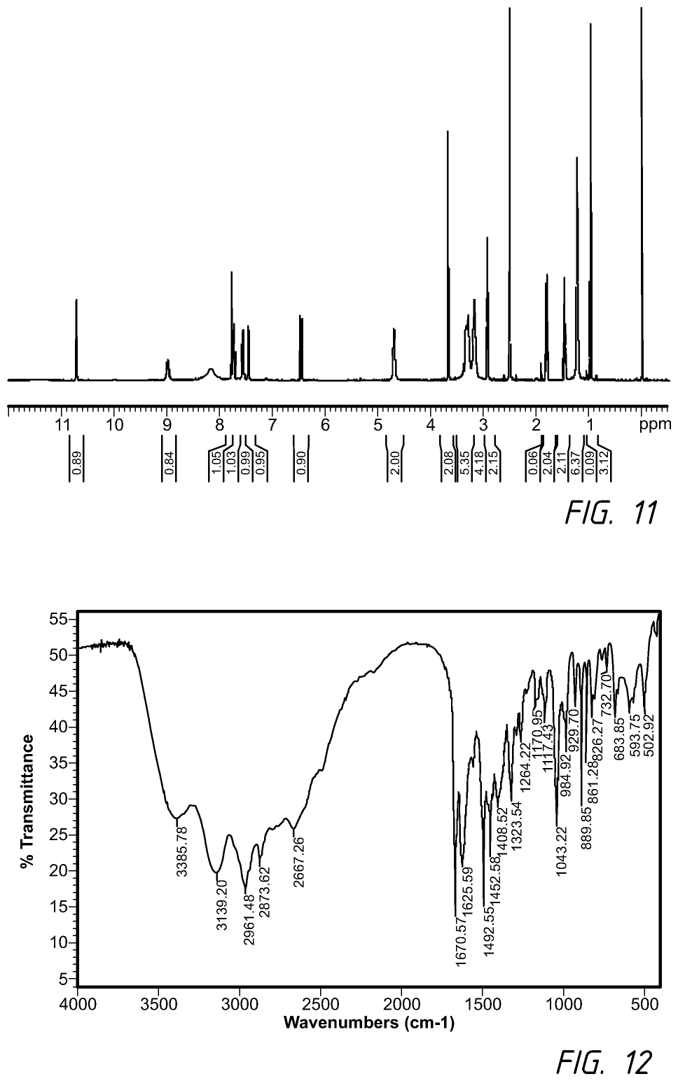

FIG. 11 provides a representative ¹H NMR of the pracinostat diHCl co-crystal with glycine. Based on the integration of 2H of pracinostat diHCl at 4.7 ppm and 2H of glycine at 3.7 ppm, the molar ratio of pracinostat diHCl to glycine is determined to be 1:1.

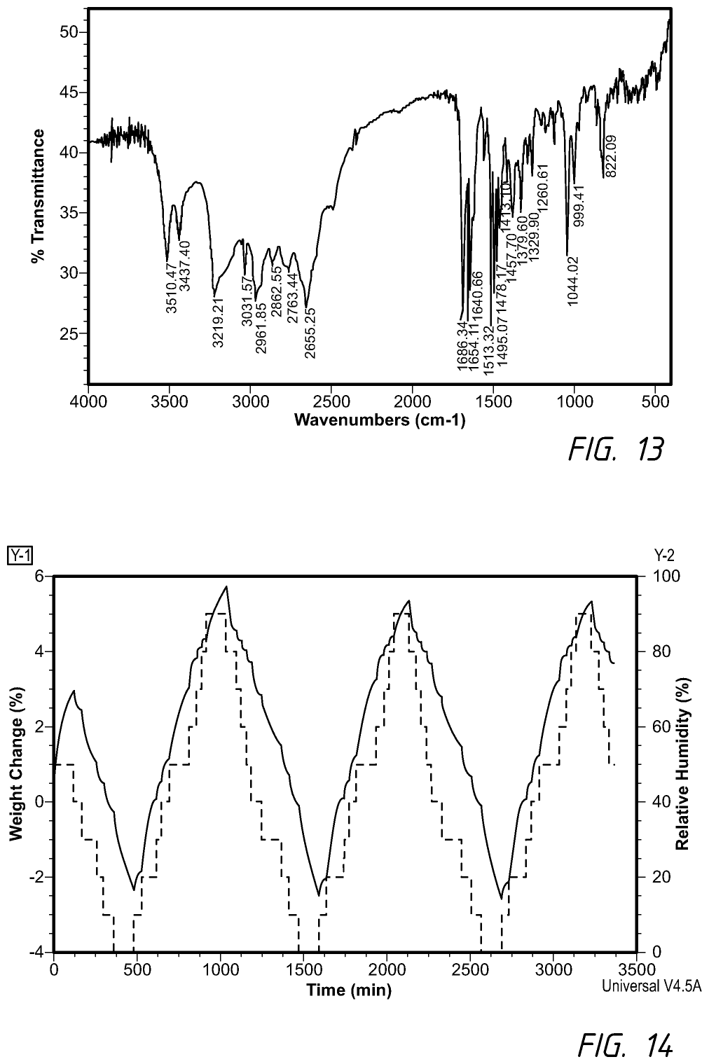

FIG. 12 provides a representative FTIR spectra of the pracinostat diHCl co-crystal with glycine. FIG. 13 provides a representative FTIR spectra of pracinostat diHCl. A comparison of FIG. 12 with FIG. 13 reveals a peak at 3385.78 cm⁻¹ which indicates that a hydrogen bond is formed, i.e., the formation of the co-crystal.

FIG. 14 provides a representative DVS plot of the pracinostat diHCl co-crystal with glycine which shows about an 8.0% weight change between 0-90% RH, indicating the sample is hygroscopic. XRPD analysis confirms that the pracinostat diHCl co-crystal with glycine remains unchanged post-DVS.

### Example 8—Preparation of Pracinostat diHCl Co-Crystal with Fumaric Acid

100 mg (0.23 mmol) of pracinostat diHCl is suspended in 2 mL of a solvent mixture of isopropyl alcohol (IPA) and water (8:1, v/v) at 40° C. 26.9 mg (0.23 mmol) fumaric acid is suspended in 5 drops (˜0.25 mL) of a solvent mixture of IPA and water (8:1, v/v) at 40° C. The pracinostat diHCl and fumaric acid samples are combined and stirred at 40° C. overnight (˜12 hr), resulting in a clear solution. The solution is cooled from 40° C. to 4° C. at the cooling rate of 0.5° C./min, resulting in a solid. The solid is isolated by vacuum filtration and analyzed by XRPD and determined to be a pracinostat diHCl co-crystal with fumaric acid.

KF measurement indicates the presence of about 5.3% (weight) water in the pracinostat diHCl co-crystal with fumaric acid, which suggests it is a hydrate, more likely, a trihydrate.

The pracinostat diHCl co-crystal with fumaric acid is characterized by its XRPD pattern peaks. 2θ and relative % intensity values for peaks are shown in Table 3.

TABLE 3
Average Peak List for Pracinostat diHCl
Co-crystal with Fumaric Acid Diffractogram

| Angle | |
|---|---|
| 2-Theta ° | Intensity % |
| 7.3 | 56.6 |
| 8.2 | 58.2 |
| 9.0 | 25.4 |
| 9.9 | 18.9 |
| 11.1 | 22.1 |
| 12.4 | 18.9 |
| 12.8 | 18.9 |
| 13.5 | 17.2 |
| 14.8 | 100 |
| 16.6 | 48.4 |
| 17.5 | 24.6 |
| 18.0 | 19.7 |
| 18.9 | 20.5 |
| 19.9 | 36.1 |
| 20.4 | 36.9 |
| 21.0 | 20.5 |
| 22.3 | 59.8 |
| 22.9 | 57.4 |
| 24.7 | 21.3 |
| 25.1 | 17.2 |
| 25.7 | 26.2 |
| 26.7 | 18.9 |
| 28.1 | 19.7 |
| 28.6 | 19.7 |
| 29.1 | 17.2 |
| 30.1 | 41 |

The angle measurements are ±0.2° 2θ. In one embodiment, key defining peaks for the pracinostat diHCl co-crystal with fumaric acid include two or more of 7.3, 8.2, 14.8, 22.3, and 22.9° 2θ. In another embodiment, key defining peaks for the pracinostat diHCl co-crystal with fumaric acid further comprise one or more peaks selected from 16.6 and 30.1σ 2θ.

Stability studies under various conditions, for example, in an open vial covered with Kimwipes and in a sealed vial, demonstrate that the pracinostat diHCl co-crystal with fumaric acid is stable for at least 9 months at 40° C./75% RH, as evidenced by XRPD.

Solubility studies are conducted on the pracinostat diHCl co-crystal with fumaric acid according to the following method:

1. Weigh out ˜6 mg pracinostat diHCl co-crystal with fumaric acid in 5 mL pH buffer;

2. Stir samples at 37° C.;

3. After 1 hour, test and record pH, then remove a 2 mL sample for HPLC analysis;

4. Adjust pH of remaining solution using 0.2M NaOH or HCl, if necessary;

5. After 4 hours, test and record pH, then remove a 2 mL sample for HPLC analysis.

The pH values and experimental observations for the pracinostat diHCl co-crystal with fumaric acid after 1 hr and 4 hrs in pH buffers are as follows:

| | | | pH | | | |
|---|---|---|---|---|---|---|
| | | | adjusted | | | |
| Exp. | Starting | pH- | after 1 | Observation- | Final | Observation- |
| # | pH | 1 hr | hr | 1 hr | pH-4 hr | 4 hr |
| 1 | 1.75 | 1.74 | — | Clear | 1.74 | Clear |
| 2 | | 1.75 | — | Clear | 1.73 | Clear |
| 3 | | 1.76 | — | Clear | 1.73 | Clear |
| 4 | 7.26 | 7.05 | 7.23 | Cloudy | 7.24 | Cloudy |
| 5 | | 7.05 | 7.30 | Cloudy | 7.28 | Cloudy |
| 6 | | 7.02 | 7.20 | Cloudy | 7.18 | Cloudy |

The solubility data for the pracinostat diHCl co-crystal with fumaric acid is contained in the chart below.

| | | | Avg. | | Avg. |
|---|---|---|---|---|---|
| | | Solu- | Solubility ± | Solu- | Solubility ± |
| | | bility | STD | bility | STD |
| Exp. | Starting | (mg/mL) | (mg/mL) | (mg/mL) | (mg/mL) |
| # | pH | 1 hr | 1 hr | 4 hr | 4 hr |
| 1 | 1.75 | >1.07 | — | >1.08 | — |
| 2 | | >0.75 | — | >0.81 | — |
| 3 | | >0.98 | — | >0.98 | — |
| 4 | 7.26 | 0.17 | 0.16 ± 0.01 | 0.09 | 0.10 ± 0.02 |
| 5 | | 0.14 | | 0.08 | |
| 6 | | 0.16 | | 0.12 | |

STD: standard deviation

The solubility data are collected using an Agilent 1100 Series HPLC system and analyzed using Chemstation software. The method details are listed below.

| Test sample make-up: | 1.0 mg/mL in acetonitrile: water (1:1, v/v) | |
|---|---|---|
| Column: | Ascends ® Express C18 | |
| | 10 cm × 4.6 mm, 2.7 μm | |
| Column Temperature (° C.): | 25 | |
| Injection (μL): | 5 | |
| Wavelength (nm): | 254 | |
| Flow Rate (mL/min): | 2.0 | |
| Phase A: | 0.1% TFA in water | |
| Phase B: | 0.085% TFA in acetonitrile | |
| Timetable: | Time (min) | % Phase B |
| | 0 | 5 |
| | 6 | 95 |
| | 6.2 | 5 |
| | 8 | 5 |

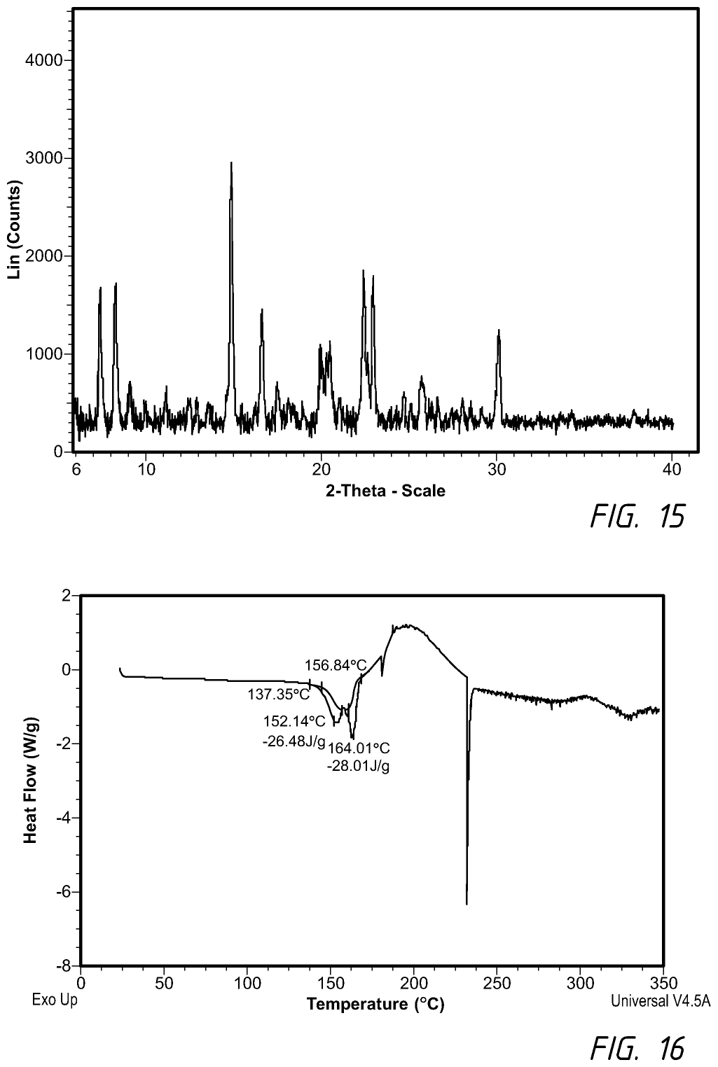

FIG. 15 provides a representative XRPD pattern for a representative sample of the pracinostat diHCl co-crystal with fumaric acid.

FIG. 16 provides a representative DSC plot of the pracinostat diHCl co-crystal with fumaric acid which shows the onset of two endothermic events, one at about 137° C. and another at about 157° C.

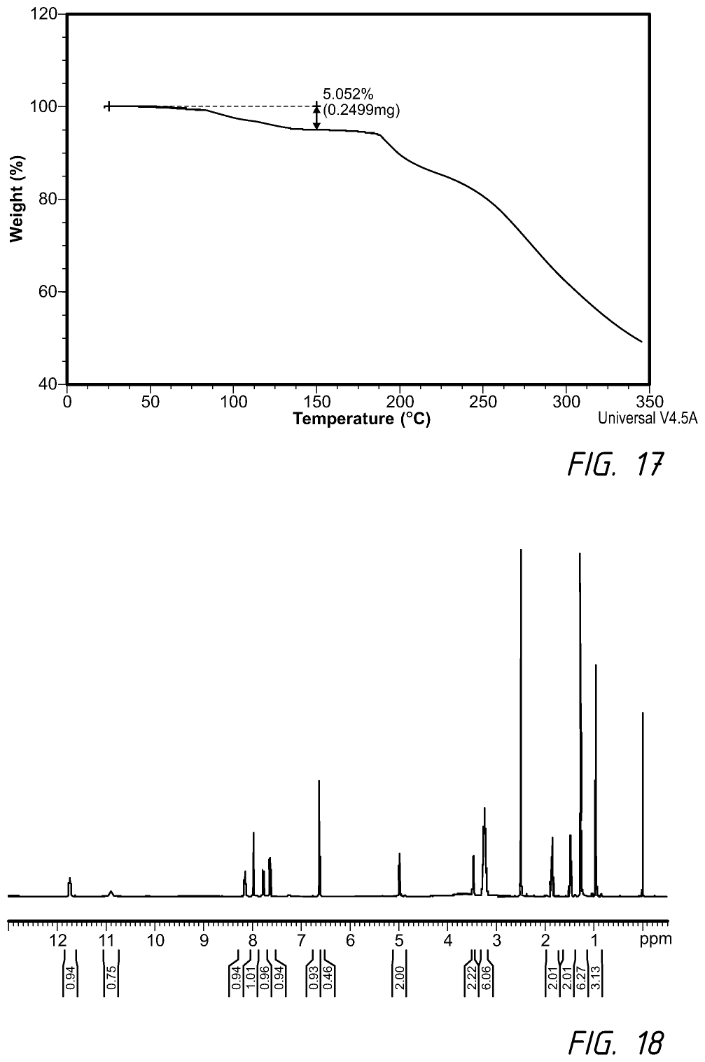

FIG. 17 provides a representative TGA plot of the pracinostat diHCl co-crystal with fumaric acid that shows a weight loss of about 5.1% up to about 150° C.

FIG. 18 provides a representative ¹H NMR of the pracinostat diHCl co-crystal with fumaric acid. Based on the integration of the 2H from fumaric acid at 6.6 ppm and 2H from pracinostat diHCl at 5.0 ppm, the molar ratio of pracinostat diHCl to fumaric acid is determined to be 2:1.

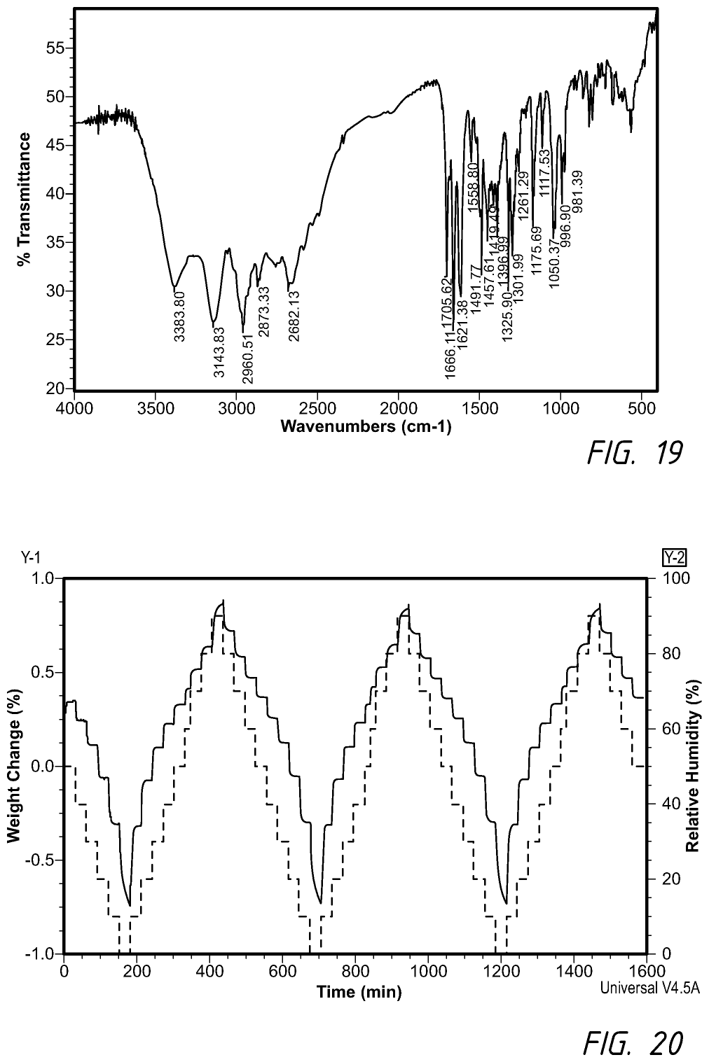

FIG. 19 provides a representative FTIR spectra of the pracinostat diHCl co-crystal with fumaric acid. A comparison of FIG. 19 with FIG. 13 (representative FTIR spectra of pracinostat diHCl) reveals a peak at 3383.8 cm⁻¹ which indicates that a hydrogen bond is formed, i.e., the formation of the co-crystal.

FIG. 20 provides a representative DVS plot of the pracinostat diHCl co-crystal with fumaric acid which shows about a 1.7% weight change between 0˜90% RH, indicating the sample is slightly hygroscopic. XRPD analysis confirms that the pracinostat diHCl co-crystal with fumaric remains unchanged post-DVS.

### Example 9—Preparation of Pracinostat diHCl Co-crystal with Fumaric Acid

2 g (4.6 mmol) of pracinostat diHCl is dissolved in 40 mL of a solvent mixture of isopropyl alcohol (IPA) and water (8:1, v/v) at 60° C. 278.6 mg (2.4 mmol) of fumaric acid solid is added to the pracinostat diHCl solution and stirred at 60° C. for about 3 hr. The solution remains clear. The sample is cooled from 60 to 5° C. at the cooling rate of 0.1° C./min. The solid precipitates at 5° C. and the solid is isolated by vacuum filtration. The solid is analyzed by XRPD which yields a pattern consistent with FIG. 15 which confirms it to be a pracinostat diHCl co-crystal with fumaric acid.

### Example 10—Preparation of Pracinostat diHCl Co-Crystal with Fumaric Acid

100 mg (0.23 mmol) of pracinostat diHCl is dissolved in 2 mL of a solvent mixture of isopropyl alcohol (IPA) and water (8:1, v/v) at 60° C. 26.9 mg (0.23 mmol) of fumaric acid solid is added to the pracinostat diHCl solution and stirred at 60° C. for about 3 hr. The solution remains clear. The sample is cooled from 60 to 5° C. at the cooling rate of 0.1° C./min. The solid precipitates at 5° C. and the solid is isolated by vacuum filtration. The solid is analyzed by XRPD which yields a pattern consistent with FIG. 15 which confirms it to be a pracinostat diHCl co-crystal with fumaric acid.

### Example 11—Preparation of Pracinostat diHCl Co-Crystal with Fumaric Acid

100 mg (0.23 mmol) of pracinostat diHCl is dissolved in 2 mL of a solvent mixture of isopropyl alcohol (IPA) and water (8:1, v/v) at 60° C. 26.9 mg (0.23 mmol) of fumaric acid is dissolved in 200 μL of a solvent mixture of IPA and water (8:1, v/v) at 60° C. The pracinostat and fumaric acid solutions are combined and stirred at 60° C. for about 3 hr. The combined solution remains clear. The sample is cooled from 60 to 5° C. at the cooling rate of 0.1° C./min. The solid precipitates at 5° C. and the solid is isolated by vacuum filtration. The solid is analyzed by XRPD which yields a pattern consistent with FIG. 15 which confirms it to be a pracinostat diHCl co-crystal with fumaric acid.

### Example 12—Preparation of Pracinostat diHCl Co-Crystal with L-Arginine

100 mg (0.23 mmol) of pracinostat diHCl is suspended in 2 mL of a solvent mixture of isopropyl alcohol (IPA) and water (8:1, v/v) at 40° C. 40.3 mg (0.23 mmol) L-arginine is suspended in 20 drops (˜1 mL) of a solvent mixture of IPA and water (8:1, v/v) at 40° C. The pracinostat diHCl and the L-arginine samples are combined and stirred for about 5 minutes, resulting in what appears to be a clear solution. The solution is stirred at 40° C. overnight (˜12 hr), resulting in the formation of a precipitate. The sample is cooled from 40° C. to 4° C. at the cooling rate of 0.5° C./min, causing the sample to freeze. The sample is transferred to room temperature causing the sample to melt. The solid is isolated by vacuum filtration and analyzed by XRPD and determined to be a pracinostat diHCl co-crystal with L-arginine.

KF measurement indicates the presence of about 1.8% (weight) water in the pracinostat diHCl co-crystal with L-arginine, which suggests it is a hydrate, more likely, a hemihydrate.

The pracinostat diHCl co-crystal with L-arginine is characterized by its XRPD pattern peaks. 2θ and relative % intensity values for peaks are shown in Table 4.

TABLE 4
Average Peak List for Pracinostat diHCl
Co-crystal with L-arginine Diffractogram

| Angle | |
|---|---|
| 2-Theta ° | Intensity % |
| 7.7 | 20.6 |
| 7.9 | 21.3 |
| 8.6 | 14.5 |
| 10.7 | 10.5 |
| 12.6 | 19.9 |
| 14.4 | 34.5 |
| 16.5 | 17.2 |
| 17.5 | 51.4 |
| 19.1 | 27 |
| 19.9 | 27 |
| 20.6 | 26 |
| 20.9 | 13.2 |
| 23.3 | 11.8 |
| 24.3 | 11.8 |
| 25.0 | 20.9 |
| 25.4 | 23 |
| 26.5 | 100 |
| 28.2 | 13.9 |
| 29.5 | 14.5 |
| 32.7 | 10.8 |
| 33.5 | 12.5 |
| 37.0 | 23.3 |

The angle measurements are ±0.2° 2θ. In one embodiment, key defining peaks for the pracinostat diHCl co-crystal with L-arginine include two or more of 14.4, 17.5, and 26.5° 2θ. In another embodiment, key defining peaks for the pracinostat diHCl co-crystal with L-arginine further comprise one or more peaks selected from 19.1, 19.9, and 20.6° 2θ.

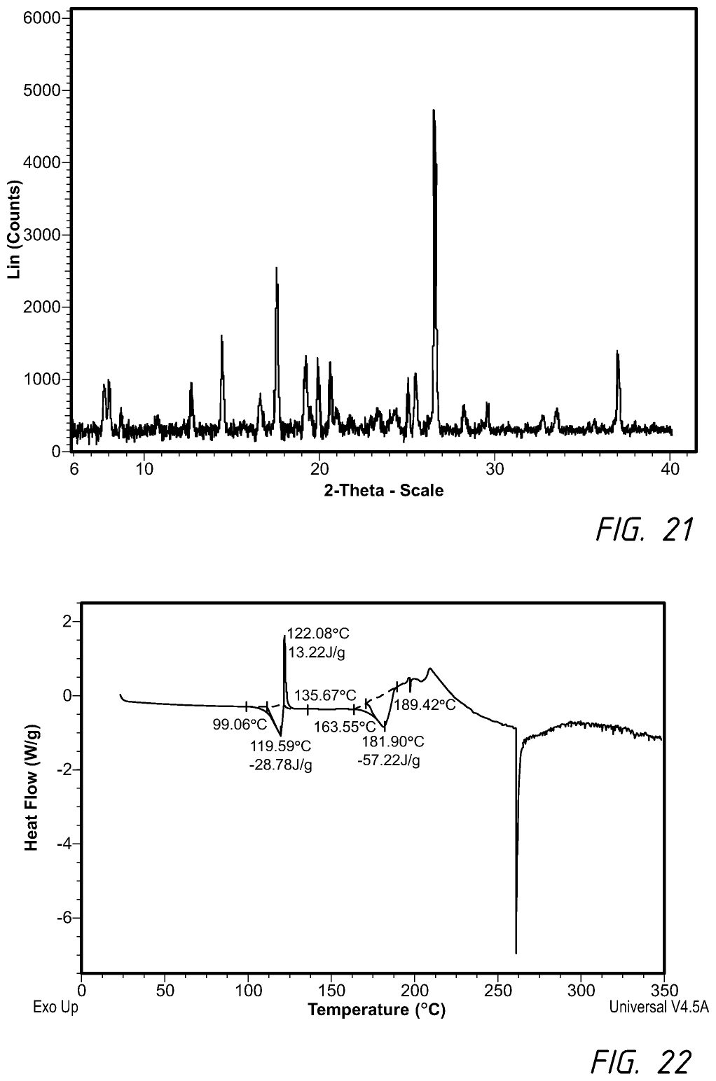

FIG. 21 provides a representative XRPD pattern for a representative sample of the pracinostat diHCl co-crystal with L-arginine.

FIG. 22 provides a representative DSC plot of the pracinostat diHCl co-crystal with L-arginine which shows the onset of two endothermic events, one at about 99° C. and another at about 164° C. and the onset of an exothermic event at about 122° C.

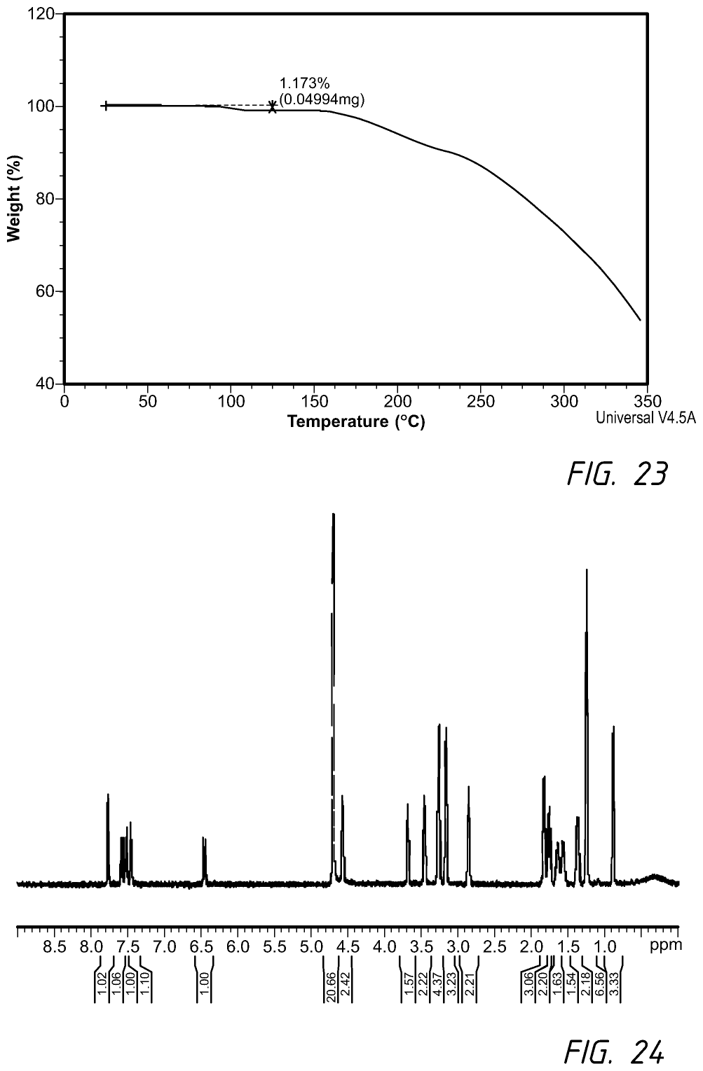

FIG. 23 provides a representative TGA plot of the pracinostat diHCl co-crystal with L-arginine that shows a weight loss of about 1.2% up to about 125° C.

FIG. 24 provides a representative ¹H NMR of the pracinostat diHCl co-crystal with L-arginine. Based on the integration of 1H of pracinostat diHCl at 6.6 ppm and 2H of L-arginine at 2.8 ppm, the molar ratio of pracinostat diHCl to L-arginine is determined to be 1:1.

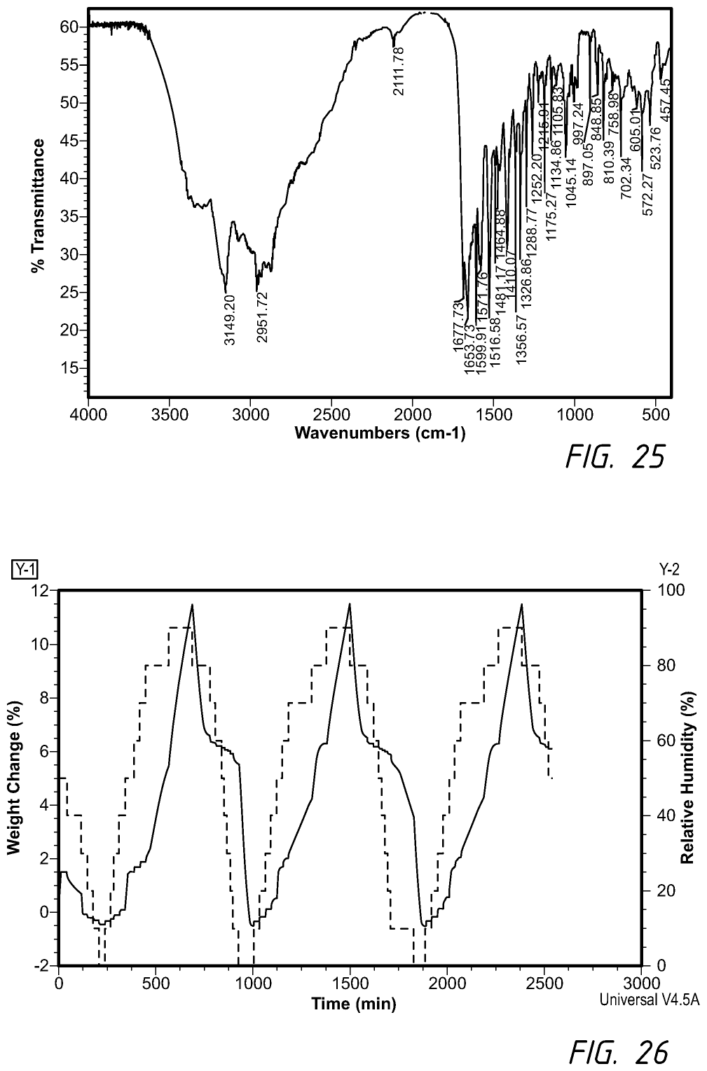

FIG. 25 provides a representative FTIR spectra of the pracinostat diHCl co-crystal with L-arginine. A comparison of FIG. 25 with FIG. 13 (representative FTIR spectra of pracinostat diHCl) reveals a hump at 3250-3400 cm⁻¹ which indicates that a hydrogen bond is formed, i.e., the co-crystal is formed.

FIG. 26 provides a representative DVS plot of the pracinostat diHCl co-crystal with L-arginine which shows about a 12% weight change between 0-90% RH, indicating the sample is hygroscopic. XRPD analysis post DVS suggests that the pracinostat diHCl co-crystal with L-arginine is not stable when exposed to certain humidity conditions.

### Example 13—Preparation of Form B of Pracinostat diHCl

566 mg (1.3 mmol) pracinostat diHCl is dissolved in 6 mL IPA (isopropyl alcohol):water (8:1, v/v) at 60° C. To 0.8 mL of this solution, about 3-5 mg Eudragit RS PO is added and the solution is cooled to 5° C. at the cooling rate of 0.1° C./min. A solid is precipitated and identified as Form B of pracinostat diHCl. KF indicates the sample contains about 6.3% (wt.) water. Form B is a hydrated form of pracinostat diHCl.

Form B is characterized by its XRPD pattern peaks. 20 and relative % intensity values for peaks are shown in Table 5.

TABLE 5
Average Peak List for Form B diffractogram

| Angle | |
|---|---|
| 2-Theta ° | Intensity % |
| 7.3 | 100 |
| 7.7 | 13.1 |
| 8.1 | 17.1 |
| 9.2 | 25.6 |
| 9.7 | 15.5 |
| 11.0 | 12.5 |
| 11.3 | 14.6 |
| 12.4 | 21.6 |
| 13.1 | 13.4 |
| 13.8 | 11.3 |
| 15.1 | 43.9 |
| 16.0 | 11.9 |
| 16.4 | 18 |
| 17.1 | 12.8 |
| 17.3 | 11 |
| 19.5 | 18.9 |
| 23.1 | 20.7 |
| 24.2 | 8.8 |
| 25.3 | 13.7 |
| 25.6 | 23.2 |
| 27.1 | 7.6 |
| 27.7 | 8.2 |
| 28.1 | 8.2 |
| 30.4 | 10.1 |

The angle measurements are 0.2° 2θ. In one embodiment, key defining peaks for Form B include 7.3, 9.2, 15.1, and 25.6° 2θ. In another embodiment, key defining peaks for Form B further comprise one or more peaks selected from 12.4 and 23.1° 2θ.

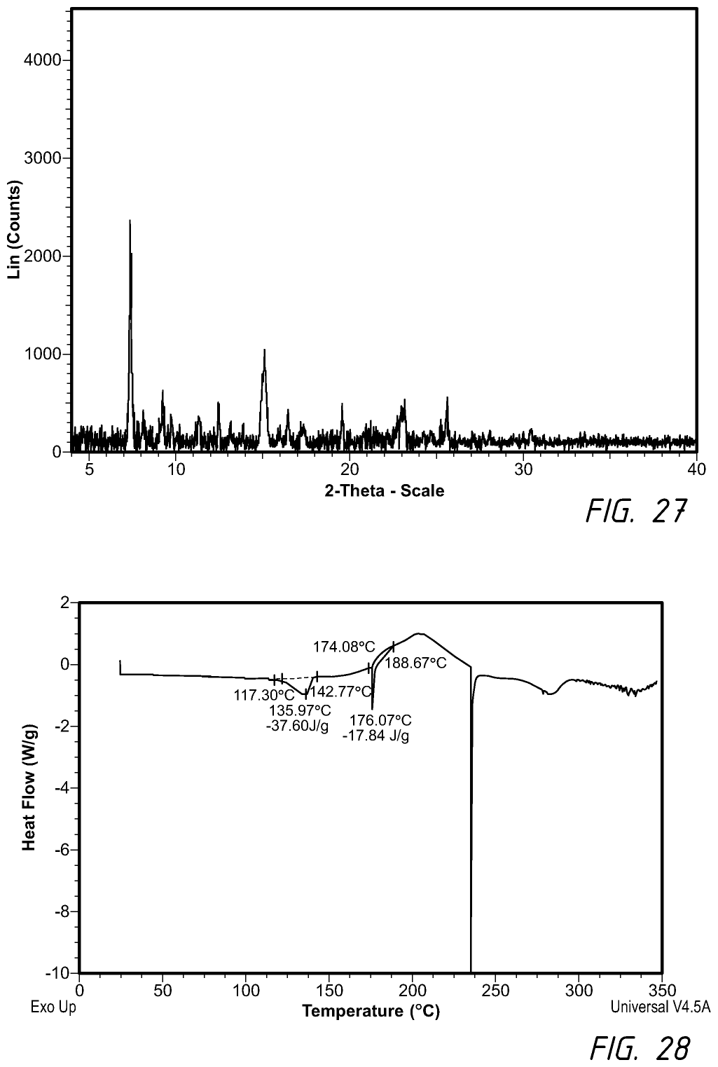

FIG. 27 provides a representative XRPD pattern for a representative sample of Form B.

FIG. 28 provides a representative DSC plot of Form B that shows the onset of two endothermic events, one at about 117° C. and the other at about 174° C.

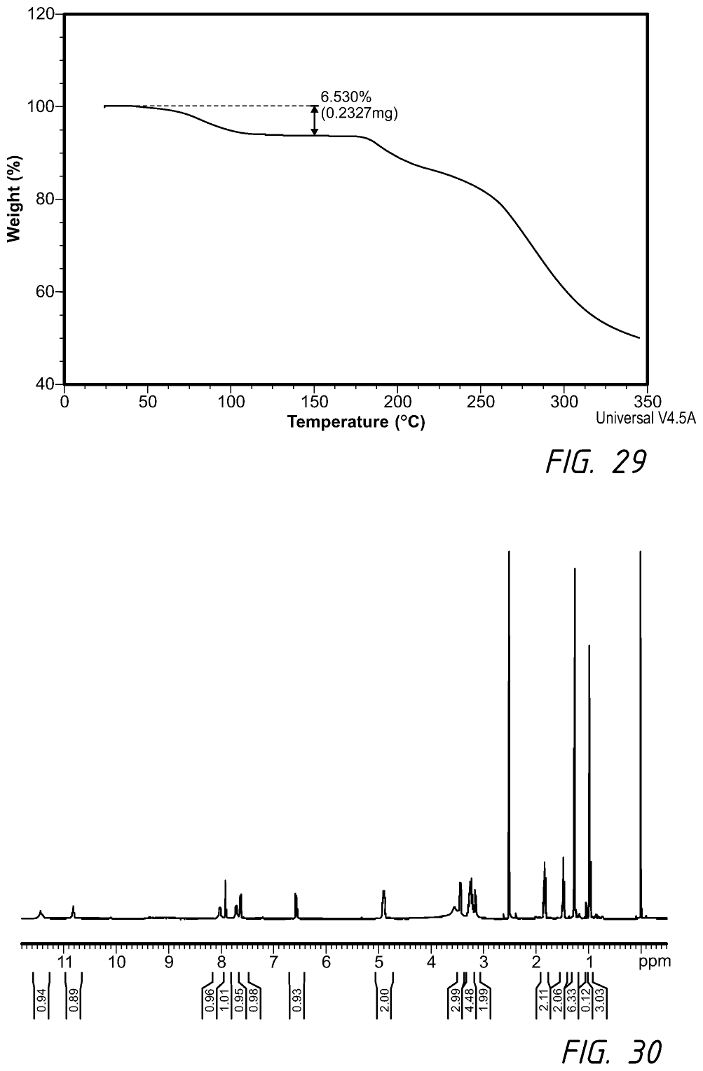

FIG. 29 provides a representative TGA plot of Form B that shows a weight loss of about 6.5% up to about 150° C.

FIG. 30 provides a representative ¹H NMR of Form B which shows the presence of residual isopropyl alcohol (0.035 equivalent, 0.5% wt.).

### Example 14—Preparation of Form B of Pracinostat diHCl

566 mg (1.3 mmol) pracinostat diHCl is dissolved in 6 mL IPA: water (8:1, v/v) at 60° C. A portion of the solution is subjected to evaporation in the lab hood until precipitation is observed. The precipitate is identified as Form B of pracinostat diHCl.

### Example 15—Preparation of Form B of Pracinostat diHCl

260 mg (0.6 mmol) pracinostat diHCl is dissolved in 1.8 mL water. 0.4 mL of this solution is stirred at 4° C. and remains a clear solution. Dioxane is added dropwise to this solution until precipitation is observed. A total of 4 mL dioxane (10 volumes) is added. The solid is isolated by vacuum filtration and identified as Form B of pracinostat diHCl.

### Example 16—Preparation of Form C of Pracinostat diHCl

602.4 mg (1.4 mmol) pracinostat diHCl is dissolved in 4 mL MeOH (methanol). 0.2 mL of this solution is placed in an open 4-mL glass vial. About 2.5 mL DCM (dichloromethane) is placed in a 20 mL glass vial. The 4 mL vial containing the pracinostat diHCl solution is placed inside the 20 mL vial which is closed to allow the vapor of DCM to interact with the MeOH solution. A solid is precipitated after 3 weeks and identified as Form C of pracinostat diHCl.

Form C is characterized by its XRPD pattern peaks. 2θ and relative % intensity values for peaks are shown in Table 6.

TABLE 6
Average Peak List for Form C diffractogram

| Angle | |
|---|---|
| 2-Theta ° | Intensity % |
| 6.3 | 28.2 |
| 6.5 | 9.2 |
| 7.7 | 16.6 |
| 7.9 | 8.5 |
| 9.6 | 8.8 |
| 13.6 | 6.1 |
| 14.8 | 14.5 |
| 18.5 | 8 |
| 18.8 | 7.7 |
| 19.5 | 100 |
| 19.7 | 14.2 |
| 20.5 | 8.5 |
| 21.9 | 5.7 |
| 22.6 | 5.1 |
| 24.3 | 12.5 |
| 24.7 | 27.6 |
| 33.3 | 6 |

The angle measurements are 0.2° 2θ. In one embodiment, key defining peaks for Form C include 6.3, 7.7, 14.8, 19.5, 19.7 and 24.7° 2θ.

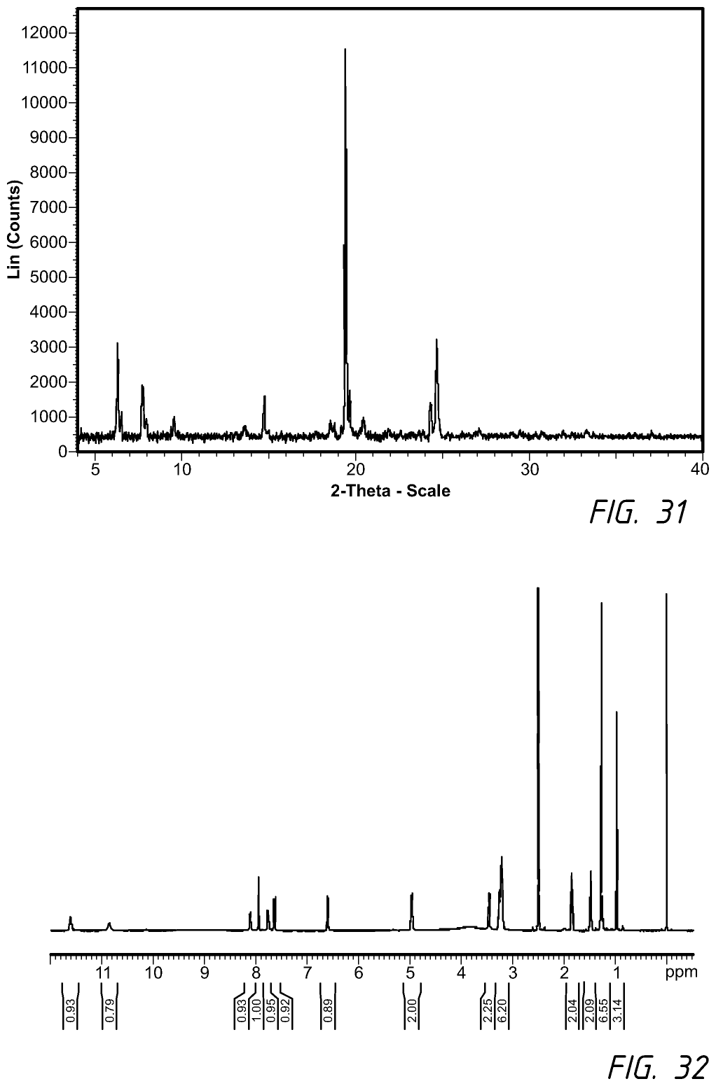

FIG. 31 provides a representative XRPD pattern for a representative sample of Form C.

FIG. 32 provides a representative ¹H NMR of Form C which shows that no residual solvent is present in the sample.

The above examples are set forth to aid in the understanding of the disclosure and are not intended and should not be construed to limit in any way the disclosure set forth in the claims which follow hereafter.

1. A pracinostat diHCl co-crystal with fumaric acid, characterized by having at least 2 or more X-ray powder diffraction peaks selected from about 7.3, 8.2, 14.8, 22.3, and 22.9° 2θ±0.2° 2θ as measured by CuKα radiation.

2. (canceled)

3. The pracinostat diHCl co-crystal with fumaric acid of claim 1, wherein the co-crystal is a hydrate.

4. (canceled)

5. (canceled)

6. The pracinostat diHCl co-crystal with fumaric acid of claim 1, which is characterized by onset of endothermic events at about 137° C. and 157° C.±3° C., as measured by differential scanning calorimetry.

7. A process for the preparation of the pracinostat diHCl co-crystal with fumaric acid according to claim 1 comprising:
a) suspending pracinostat diHCl in a solvent mixture of isopropyl alcohol and water;
b) suspending fumaric acid in a solvent mixture of isopropyl alcohol and water, wherein the molar ratio of pracinostat diHCl to fumaric acid is about 1:1;
c) mixing the pracinostat diHCl and fumaric acid suspensions to yield a clear solution of pracinostat diHCl and fumaric acid; and
d) cooling the solution of pracinostat diHCl and fumaric acid to yield the pracinostat diHCl co-crystal with fumaric acid.

8. (canceled)

9. (canceled)

10. (canceled)

11. A process for the preparation of the pracinostat diHCl co-crystal with fumaric acid according to claim 1 comprising:
a) combining pracinostat diHCl with a solvent mixture of isopropyl alcohol and water at an elevated temperature to form a pracinostat diHCl solution;
b) adding solid fumaric acid to the pracinostat diHCl solution;
c) stirring the mixture of fumaric acid and pracinostat diHCl solution of step b at the elevated temperature;
d) cooling the mixture of step c to yield the pracinostat diHCl co-crystal with fumaric acid.

12.-17. (canceled)

18. A process for the preparation of the pracinostat diHCl co-crystal with fumaric acid according to claim 1 comprising:
a) combining pracinostat diHCl with a solvent mixture of isopropyl alcohol and water at an elevated temperature to form a pracinostat diHCl solution;
b) combining fumaric acid with a solvent mixture of isopropyl alcohol and water at the elevated temperature to form a fumaric acid solution, wherein the molar ratio of pracinostat diHCl to fumaric acid is about 1:1;
c) combining the pracinostat diHCl solution and the fumaric acid solution;
d) stirring the combined solution of step c at the elevated temperature;
e) cooling the combined solution to yield the pracinostat diHCl co-crystal with fumaric acid.

19.-74. (canceled)

75. Form B of pracinostat diHCl which is characterized by having X-ray powder diffraction peaks at about 7.3, 9.2, 15.1, and 25.6° 2θ±0.2° 2θ as measured by CuKα radiation.

76. (canceled)

77. Form B of pracinostat diHCl of claim 75 which is a hydrate.

78. Form B of pracinostat diHCl according to claim 75, which is characterized by onset of endothermic events at about 117° C. and at about 174° C.±3° C., as measured by differential scanning calorimetry.

79. A process for the preparation of Form B of pracinostat diHCl according to claim 75 comprising:
a) combining pracinostat diHCl with a solvent mixture of isopropyl alcohol and water to obtain a solution of pracinostat diHCl;
b) adding Eudragit RS PO to the solution of pracinostat diHCl; and
c) cooling the solution of pracinostat diHCl with Eudragit RS PO of step b to yield Form B of pracinostat diHCl as a solid.

80.-90. (canceled)

91. Form C of pracinostat diHCl which is characterized by having X-ray powder diffraction peaks at about 6.3, 7.7, 14.8, 19.5, 19.7 and 24.7° 2θ±0.2° 2θ as measured by CuKα radiation.

92. A process for the preparation of Form C of pracinostat diHCl according to claim 91 comprising:
a) combining pracinostat diHCl and methanol to obtain a solution of pracinostat diHCl; and
b) allowing vapor diffusion to occur between dichloromethane and the solution of pracinostat diHCl to yield Form C of pracinostat diHCl as a solid.

93. (canceled)

94. (canceled)

95. (canceled)
